(19) United States
(12) Patent Application Publication
(10) Pub. No.: US 2026/0232890 A1
(43) Pub. Date: Aug. 13, 2026

(54) Filter Assembly For Filtering Smoke Evacuated With A Medical Waste Collection System Including A Vacuum

(71) Applicant: Stryker Corporation, Portage, MI (US)

(72) Inventors: Joshua Caleb Colvin, Paw Paw, MI (US); Dharmendra Kumar, Ghaziabad (IN); Anshul Shandillya, Agra (IN); Dinesh Sharma, Faridabad (IN)

(73) Assignee: Stryker Corporation, Portage, MI (US)

(21) Appl. No.: 19/154,289

(22) PCT Filed: Feb. 7, 2024
(86) PCT No.: PCT/US2024/014877

§ 371 Date: Aug. 6, 2025

Related U.S. Application Data
(60) Provisional application No. 63/483,750, filed on Feb. 7, 2023.

(51) Int. Cl. A61M 1/00; A61B 18/00

(52) U.S. Cl. CPC A61M 1/79; A61B 18/00; A61M 1/74; A61M 1/84; A61B 2218/008; A61M 2205/3317; A61M 2205/502

(57) ABSTRACT

Systems and methods for evacuation of surgical smoke. A capital component may include a vacuum source, and a controller. A filter assembly is configured to be removably coupled to the capital component and include a housing defining a socket for connecting a smoke evacuation tube, and include a flap for selectively covering the socket. The sensor is configured to detect the position of the flap relative to the socket. The controller may be configured to operate the system based on the position of the flap relative to the socket, as detected by the sensor. The controller may be configured to enable disable, or adjust the operation of the vacuum source based on the signal, and/or open or close a valve associated with the vacuum source. Assemblies and methods for removing filter media from the housing of the filter assembly are also disclosed.

### CROSS-REFERENCE TO RELATED APPLICATIONS

The present application claims priority to and all the benefits of U.S. Provisional Patent Application No. 63/483,750, filed on Feb. 7, 2023, the entire contents of which are expressly incorporated herein by reference.

### BACKGROUND

Various medical devices incorporate filtration systems intended to maintain sterilization and/or for capturing hazardous particles. For example, a medical device including a vacuum may include a filtration system for removing debris and/or hazardous particles from the liquid or air that is consumed by the vacuum. Depending on the type of medical device, the filtration system may require a large and/or complex filter assembly to achieve filtration of particulates.

Similarly the filtrations systems may include a filter assembly including one or more sockets or ports through which a tubing of a surgical instrument may be connected. The sockets and ports may be configured to allow debris and/or hazardous particles to enter the filter assembly. A vacuum or suction may be applied to the filter assembly to draw debris and/or hazardous particles into the filter assembly through the sockets or ports. The filter assembly may further comprise a lid or flap intended to cover the socket or port when the socket or port is not in use and/or a surgical instrument is not attached. The operation of the filtration system, and more specifically operation of a vacuum source for applying the vacuum or suction to the filter assembly, may vary based on whether the socket or port is exposed/uncovered and/or an instrument is attached to one or more of the socket(s) or port(s). Presently conventional filter assembly do not provide or allow for identification of a flap position indicating whether the socket(s) or port(s) is exposed or covered and/or indication of whether a surgical instrument is attached to the filter assembly. This information could be utilized to optimize the operation of the filtration system.

Therefore, there remains a need in the art for a filter assembly configured to solve various disadvantages of conventional filter assemblies.

### SUMMARY

The present disclosure relates generally to a filter assembly and method of using the filter assembly with a medical device, such as a medical waste collection system or surgical smoke evacuator that includes a vacuum. The filter assembly comprises a filter cartridge that may be configured to filter smoke and other particles collected by the vacuum of the medical waste collection system.

A filter assembly for use with a medical waste collection system may include a first configuration of a filter cartridge. The medical waste collection system may comprise a handpiece removably coupled to the filter assembly by a smoke evacuation tube. The filter assembly may comprise an housing defining an interior configured to receive the filter cartridge. The housing may comprise a cover and a sensor. The first configuration of the filter cartridge may comprise a faceplate having an inner surface and an opposing outer surface. The filter cartridge may further comprise a first alignment feature coupled to a perimeter of the faceplate, wherein the first alignment feature may be configured to orient the filter cartridge within the interior of the housing. The filter cartridge may also comprise a filter portion having a first end and a second end, wherein the first end of the filter portion is spaced apart from the inner surface of the faceplate to at least partially define a void space between the faceplate and the filter portion. The faceplate may comprise a first port and a second port each configured to receive an end of the smoke evacuation tube(s) that is opposite the handpiece. Each of the ports may be positioned on the faceplate to be in communication with the void space and configured to allow smoke to pass through the faceplate and enter the void space. The first port may comprise a first dimension and the second port may comprise a second dimension to allow for smoke evacuation tubes of varying dimensions to be removably coupled to the faceplate. The faceplate may further comprise a sensor opening, wherein the sensor opening may be configured to receive the sensor of the housing when the filter cartridge is disposed within the interior of the housing such that the sensor is at least partially disposed within the void space of the filter cartridge.

A filter assembly for use with a medical waste collection system may include a second configuration of a filter cartridge for filtering smoke. The filter assembly may include a filter assembly housing including a front cover at least partially defining an interior, and a sensor assembly including a sensor housing and a sensor. The second configuration of the filter cartridge may comprise a faceplate configured to be positioned within the interior of the filter assembly housing near the front cover with the faceplate comprising an outer surface opposite an inner surface and defining a port configured to be removably coupled with a smoke evacuation tube. The faceplate may also comprise a sensor opening separate from the port with the sensor opening sized to receive at least a portion of the sensor housing of the sensor assembly. The filter cartridge may further comprise a filter portion comprising a front, a rear opposite the front, and a side extending between the front and the rear. The front of the filter portion may be directed towards the inner surface of the faceplate. The faceplate and the filter portion may be spaced apart from one another to at least partially define a void space between the inner surface of the faceplate and the front of the filter portion. The sensor opening and the port of the faceplate may be complementarily arranged and in communication with the void space such that, when the filter cartridge is disposed within the interior of the filter assembly housing and the smoke evacuation tube is removably coupled with the port, the sensor at least partially disposed within the void space to detect smoke within the void space that is received from the smoke evacuation tube through the port before encountering the filter portion.

A first general aspect of a filter assembly system for use with a surgical smoke evacuation system may comprise an housing defining an interior. The housing may comprise a cover, wherein a sensor may be coupled to the cover and disposed within the interior of the housing. The filter assembly may further comprise a filter cartridge removably disposed within the interior of the housing. The filter cartridge may comprise a faceplate and a filter portion that is spaced apart from the faceplate to at least partially define a void space between the faceplate and the filter portion. The faceplate may comprise a first port in communication with the void space and configured to be removably coupled with a smoke evacuation tube to allow material to pass through the faceplate and enter the void space. The faceplate may further comprise a sensor opening, wherein the sensor opening may be configured to receive the sensor when the filter cartridge is disposed within the interior of the housing such that the sensor is at least partially disposed within the void space and configured to detect the presence of material entering the void space through the first port.

A second general aspect of a filter assembly system for use with a surgical smoke evacuation system may comprise an housing defining an interior, wherein the housing comprises a cover. The cover may comprise at least one aperture defining a pathway from the interior of the housing to an exterior of the housing. The filter assembly may further comprise a filter cartridge removably disposed within the interior of the housing. The filter cartridge may comprise a faceplate positioned adjacent the cover when the filter cartridge is removably disposed within the interior. The faceplate may comprise an inner surface and an opposing outer surface. The filter cartridge may further comprise a filter portion that is spaced apart from the faceplate to at least partially define a void space between the inner surface of the faceplate and the filter portion. The faceplate may also comprise a first port in communication with the void space and configured to be removably coupled with a smoke evacuation tube to allow material to pass through the faceplate and enter the void space. The faceplate may further comprise an annular boss encircling the first port and extending distally from the outer surface of the faceplate. The annular boss may be configured to create a seal between the smoke evacuation tubes and the faceplate to ensure all particles passing through the smoke evacuation tubes are collected within the filter cartridge. The annular boss encircling the first port may be configured to be at least partially disposed within one of the at least one apertures in the cover, such that a distal end of the annular boss is positioned distally of the cover (complementary aperture).

A third general aspect of a filter assembly system for use with a surgical smoke evacuation system may comprise an housing defining an interior, wherein the housing may comprise a cover including an interior surface and an exterior surface. The filter assembly may further comprise a filter cartridge removably disposed within the interior of the housing. The filter cartridge may comprise a faceplate comprising an inner surface and an opposing outer surface positioned adjacent to the cover of the housing when the filter cartridge is removably disposed within the interior of the housing. The outer surface of the filter cartridge may comprise a first portion and a second portion. The filter cartridge may also comprise a filter portion spaced apart from the faceplate to at least partially define a void space between the inner surface of the faceplate and the filter portion. The cover may also comprise a protrusion extending proximally from the interior surface. The first surface of the faceplate may be positioned distally from the second surface of the faceplate to define a recess in the outer surface of the faceplate that is configured to receive the protrusion on the interior surface of the cover when the filter cartridge is removably disposed within the interior of the housing.

The disclosure further relates to a method of replacing a filter cartridge of a filter assembly for use with a surgical medical waste collection system. The filter assembly may comprise an housing defining an interior, an opening in the housing, and a groove or slot. The slot may be defined by the opening, or the groove may be defined by an interior surface of the housing. The filter assembly may also comprise a front cover having an aperture and a sensor disposed within the housing. The method of replacing a filter cartridge of a filter assembly may comprise the step of providing the filter cartridge, wherein the filter cartridge comprises: a faceplate having a port to removably couple a smoke evacuation tube to the faceplate, an alignment feature coupled to an exterior of the cartridge, and a sensor opening in the faceplate. The method may further comprise the step of orienting the filter cartridge so the alignment feature of the filter cartridge may be inserted within the groove on the interior surface of the housing prior to sliding the filter cartridge through the opening. The method may also comprise installing the filter cartridge within the housing by sliding the filter cartridge through the opening so that the alignment feature is disposed within the groove, the sensor is at least partially disposed within the sensor opening of the filter cartridge, and the port of the filter cartridge is aligned with the aperture in the cover. The method may further comprise installing the filter assembly within the surgical medical waste collection system, such as a surgical smoke evacuation system.

A fourth general aspect of a filter assembly also includes a vacuum source; a smoke evacuation tube; a filter assembly, the filter assembly may include: a housing defining a socket for removably receiving a smoke evacuation tube; a filter disposed within an interior of the housing; a vacuum source configured to draw suction on the socket to draw fluid through the filter; a flap coupled to the housing and configured to be moved from a first position in which the flap covers the socket to a second position in which the socket is exposed to allow coupling of the smoke evacuation tube with the socket; a sensor coupled to the housing and configured to detect the flap in the first position. The unit also includes a controller in electronic communication with the sensor and configured to disable operation of the vacuum source based on the sensor detecting the flap in the first position.

A fifth general aspect of a method of operating a filter assembly including a vacuum pump. The method also includes receiving, with the controller, a signal from the sensor with the signal indicative that the flap has been moved away from covering the socket. The method also includes enabling, with the controller, operation of the vacuum pump based on the signal.

A sixth general aspect of a method of operating a filter assembly including a vacuum pump. The method also includes receiving, with the controller, a signal from the sensor with the signal indicative that the flap is covering the socket. The method also includes preventing, with the controller, operation of the vacuum pump based on the signal.

A seventh general aspect of a non-transitory computer readable medium storing computer readable instructions which. The non-transitory computer readable medium storing computer readable instructions also includes receive a first signal from a sensor of a surgical filter assembly, the sensor positioned on a housing of the surgical filter assembly proximate a socket defined in the housing, the sensor configured to generate a second signal indicating a flap is in a first position covering the socket or in a second position exposing the socket. The instructions also includes transmit the second signal to a controller in communication with a vacuum source configured to draw a vacuum through the socket when actuated, the controller configured to enable operation of the vacuum source when the second signal indicates the flap is in the second position and to disable operation of the vacuum source when the second signal indicates the flap is in the first position.

An eighth general aspect of a filter assembly also includes a housing defining a first socket for removably receiving a smoke evacuation tube. The unit also includes a filter disposed within an interior of the housing. The unit also includes a vacuum source configured to draw suction on the first socket to draw fluid through the filter. The unit also includes a first flap coupled to the housing and configured to be moved from a first position in which the first flap covers the first socket to a second position in which the first socket is exposed to allow coupling of the smoke evacuation tube with the first socket. The unit also includes a sensor coupled to the housing and configured to detect the first flap in the first position. The unit also includes a user interface. The unit also includes a controller in electronic communication with the sensor and the user interface, the controller configured to determine the first flap is in a second position based on an absence of a signal being received from the sensor and to cause the user interface to display information based on the absence of the signal.

A ninth general aspect of a filter assembly also includes a housing defining a first socket and a second socket, each of the sockets for removably receiving a smoke evacuation tube. The unit also includes a filter disposed within an interior of the housing. The unit also includes a vacuum source configured to draw suction on the socket to draw fluid through the filter. The unit also includes a first flap coupled to the housing and configured to be moved from a first position in which each of the first flap covers the first socket to a second position in which the first socket is exposed to allow coupling of the smoke evacuation tube with the first socket. The unit also includes a second flap coupled to the housing and configured to be moved from a first position in which each of the second flap covers the second socket to a second position in which the second socket is exposed to allow coupling of the smoke evacuation tube with the second socket. The unit also includes a sensor coupled to the housing and configured to detect each of the first and second flaps in the first position. The unit also includes a controller in electronic communication with the sensor and configured to identify the exposed socket based on a signal from the sensor indicative of the respective positions of the first or the second flaps.

A tenth general aspect of a filter assembly also includes a compartment defining a void. The unit also includes a housing removably coupled to the compartment and defining a socket for removably receiving a smoke evacuation tube. The unit also includes a filter disposed within an interior of the housing. The unit also includes a vacuum source at least partially disposed within the interior of the compartment and configured to draw suction on the socket to draw fluid through the filter. The unit also includes a valve disposed on the compartment, the valve moveable between a closed position in which the void is sealed from an external environment and an open position in which the void is in fluid communication with the external environment. The unit also includes a flap coupled to the housing and configured to be moved from a first position in which the flap covers the socket to a second position in which the socket is exposed to allow coupling of the smoke evacuation tube with the socket. The unit also includes a sensor coupled to the housing and configured to detect the flap in the first position. The unit also includes a controller in electronic communication with the sensor and configured to at least partially open the valve based on the sensor detecting the flap in the first position to provide ambient air from the external environment to the void to cool the vacuum source.

An eleventh general aspect includes a filter assembly for use with a surgical instrument. The filter assembly also includes a housing defining a first socket and a second socket for removably receiving a smoke evacuation tube of the surgical instrument. The unit also includes a filter disposed within an interior of the housing. The unit also includes a vacuum source configured to draw suction on the socket to draw fluid through the filter. The unit also includes a first flap coupled to the housing and configured to be moved from a first position in which each of the first flap covers the first socket to a second position in which the first socket is exposed to allow coupling of the smoke evacuation tube with the first socket. The unit also includes a second flap coupled to the housing and configured to be moved from a first position in which each of the second flap covers the second socket to a second position in which the second socket is exposed to allow coupling of the smoke evacuation tube with the second socket. The unit also includes a sensor coupled to the housing and configured to detect when either of the first or second flap is in the first position. The unit also includes a controller in electronic communication with the sensor and configured to control operation of the vacuum source based on the first or second flap being in one of the first position or the second position.

A twelfth general aspect includes a filter assembly for use with a surgical instrument is described. The filter assembly also includes a housing defining a socket for removably receiving a smoke evacuation tube. The unit also includes a filter disposed within an interior of the housing. The unit also includes a flap coupled to the housing and configured to be moved from a first position in which the flap covers the socket to a second position in which the socket is exposed to allow coupling of the smoke evacuation tube with the socket. The unit also includes a sensor coupled to the housing and configured to detect the flap in the first position.

A thirteenth general aspect includes a filter assembly for use with a vacuum source configured to draw suction is described. The filter assembly also includes a housing defining a first socket and a second socket, each of the sockets for removably receiving a smoke evacuation tube. The unit also includes a filter disposed within an interior of the housing. The unit also includes a first flap coupled to the housing and configured to be moved from a first position in which each of the first flap covers the first socket to a second position in which the first socket is exposed to allow coupling of the smoke evacuation tube with the first socket. The unit also includes a second flap coupled to the housing and configured to be moved from a first position in which each of the second flap covers the second socket to a second position in which the second socket is exposed to allow coupling of the smoke evacuation tube with the second socket. The unit also includes a sensor coupled to the housing and configured to detect each of the first and second flaps in the first position.

In some implementations, the filter assembly may optionally include a vacuum source configured to draw suction on the first socket to draw fluid through the filter. The unit may also optionally include a controller in electronic communication with the sensor. The controller may be configured to disable operation of the vacuum source based on the sensor detecting the flap in the first position.

In some implementations, the filter of the filter assembly may optionally comprise a faceplate having an inner surface and an opposing outer surface. The filter may also include a first alignment feature coupled to the faceplate, the first alignment feature configured to orient the filter within the interior of the housing. Where the faceplate comprises a port in fluid communication with the socket of the housing, the port may optionally comprises an annular boss that encircles the port and extends distally from the outer surface of the faceplate, the annular boss configured to create a seal between each of the smoke evacuation tube and the faceplate to ensure all particles passing through the smoke evacuation tube is collected within the filter.

In some implementations, the filter assembly having a vacuum source may also include a compartment defining a void. The vacuum source may be at least partially disposed within the void. A valve may be disposed on the compartment and to selectively open and close a passage defined between the void and the external environment. The valve may be in electronic communication with the controller, wherein the controller is further configured to at least partially open the valve based on the sensor detecting the flap in the first position.

These and other configurations, features, and advantages of the present disclosure will be apparent to those skilled in the art. The present disclosure is not to be limited to or by these configurations, features, and advantages.

### BRIEF DESCRIPTION OF THE DRAWINGS

Referring now to the drawings, exemplary illustrations are shown in detail. The drawings may not necessarily be to scale and certain features may be exaggerated to better illustrate and explain an innovative aspect of the present disclosure. Further, the exemplary illustrations described herein are not intended to be exhaustive or otherwise limiting or restricting to the precise form and configuration shown in the drawings and disclosed in the following detailed description.

Advantages of the present disclosure will be readily appreciated as the same becomes better understood by reference to the following detailed description when considered in connection with the accompanying drawings.

FIG. 1 is a perspective view of a filter assembly including an housing and a front cover.

FIG. 2 is a perspective view of the front cover of the filter assembly of FIG. 1.

FIG. 3 is a perspective view of an aperture cover for use with the front cover of the filter assembly, the aperture cover including one or more flaps for covering an aperture in the front cover.

FIG. 4 is a rear elevation view of the housing and front cover of FIG. 1.

FIG. 5A is a perspective view of a filter cartridge including a faceplate and cartridge cover, the filter cartridge configured to be removably disposed within the housing of the filter assembly of FIG. 1.

FIG. 5B is a sectional view of the filter cartridge of FIG. 5A.

FIG. 5C is a front view of the filter cartridge of FIG. 5A.

FIG. 6 is a rear perspective view of the faceplate of the filter cartridge of FIG. 5A.

FIG. 7 is a side elevation view of the filter cartridge of FIG. 5A with the cartridge cover removed to expose the internal features of the filter cartridge.

FIG. 8 is a perspective view of the filter assembly of FIG. 1 with the front cover removed to expose the position and orientation of the filter cartridge of FIG. 5A within the housing.

FIG. 9 is a sectional view of the filter assembly of FIG. 1 taken along section lines 9-9 and including the filter cartridge of FIG. 5A disposed within the housing.

FIG. 10 is a partially exploded view of the filter assembly of FIG. 1 including the filter cartridge of FIG. 5A.

FIG. 11A is an alternative partially exploded view of the filter assembly of FIG. 1 including the filter cartridge configuration of FIG. 5A.

FIG. 11B is an alternative partially exploded view of the filter assembly of FIG. 1 including an alternate filter cartridge configuration.

FIG. 12 is a schematic diagram illustrating the method of inserting and removing the filter assembly of FIG. 1 from a medical device or system.

FIG. 13 is a schematic diagram illustrating the various internal components of the medical device or system of FIG. 12, the internal components including at least a controller and a vacuum source for pulling a vacuum through the filter assembly.

FIG. 14 is a partially exploded view of the vacuum source of the medical device or system of FIGS. 12 and 13.

FIG. 15 is a sectional view of the vacuum source of FIG. 14.

FIG. 16A is a perspective view of an alternative configuration of a filter assembly for use with of the medical device or system of FIGS. 12 and 13, the filter assembly including an housing with a front cover defining a socket, a flap for selectively covering the socket, and a sensor for sensing the position of the flap in a first configuration.

FIG. 16B is a perspective view of an alternative configuration of a filter assembly for use with of the medical device or system of FIGS. 12 and 13, the filter assembly including an housing with a front cover defining a socket, a flap for selectively covering the socket, and a sensor for sensing the position of the flap in a second configuration.

FIG. 17A is a perspective view of the filter assembly of FIG. 16, the filter assembly illustrating a flap in a second position exposing a socket defined by the cover.

FIG. 17B is a perspective view of the filter assembly of FIG. 16, the filter assembly illustrating two flaps in a second position exposing two corresponding sockets defined by the cover.

### DETAILED DESCRIPTION

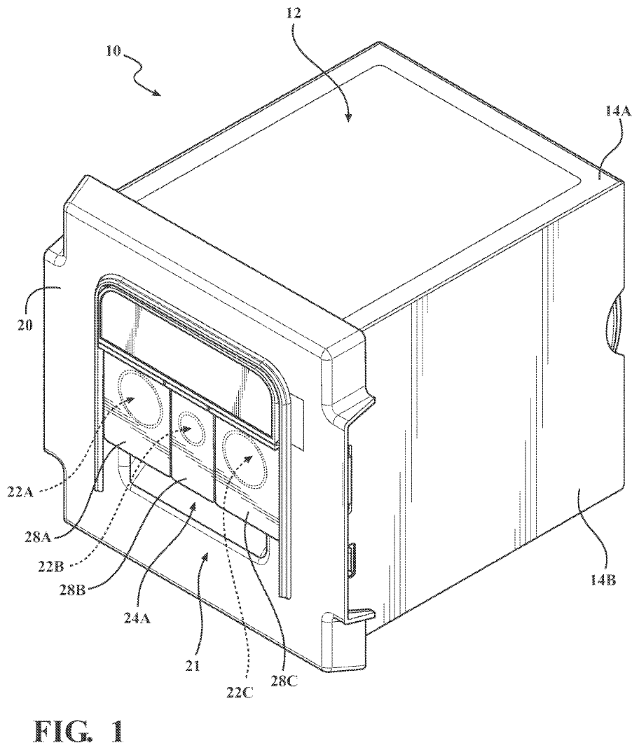

As medical professionals strive for reducing the amount of waste produced by execution of various surgical or medical procedures, they look for opportunities to reduce the number of disposable components and increase the number of reusable components. When attempting to identify and design a component of a medical device and/or instrument that can be reused, a number of factors must be considered. Specifically, the design and/or function of the reusable component must allow for sterilization of the environment and the medical devices or instruments. One such component that may be used with a medical device and has traditionally been a disposable component is a filter assembly. For example, a filter assembly may be used with a medical waste collection system 100 including a vacuum, such as a smoke evacuation system, for filtering various particles, smoke, and/or fluid that are collected from the air during the medical procedure. Traditionally, the entire filter assembly has been disposed following each individual medical procedure. Therefore, there remains a need in the art for a filter assembly that includes a combination of reusable and disposable components, such as the filter assembly 10 illustrated in FIG. 1.

Referring to FIG. 1, a perspective view of an example configuration of a filter assembly 10 is illustrated. Accordingly, the filter assembly 10 may comprise an housing 12. The housing may also be referred to as a housing, filter housing, compartment, or the like. The housing 12 may comprise a plurality of wall members 14A, 14B configured to define an interior 16 (not illustrated in FIG. 1) of the housing 12. The wall members 14 may be constructed from a generally rigid material, such as a plastic polymer or metallic alloy. While the housing 12 illustrated in FIG. 1 comprises four wall members 14A, 14B, 14C, 14D arranged in a square or rectangular configuration, it is contemplated that the size, shape, and/or number of wall members 14 of the housing 12 may vary. For example, while not illustrated in the figures, the housing 12 may comprise three wall members 14 to define a triangular-shaped housing 12. Alternatively, the housing 12 may comprise a single wall member 14 configured to define a circular- or oval-shaped housing 12.

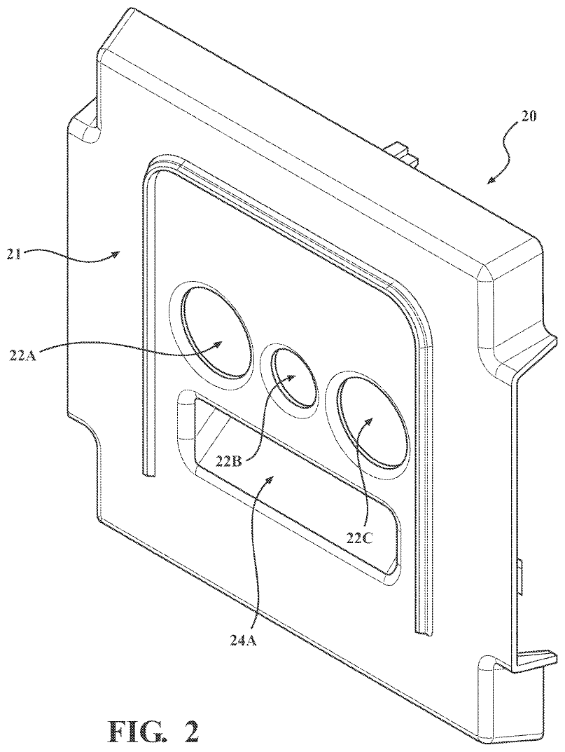

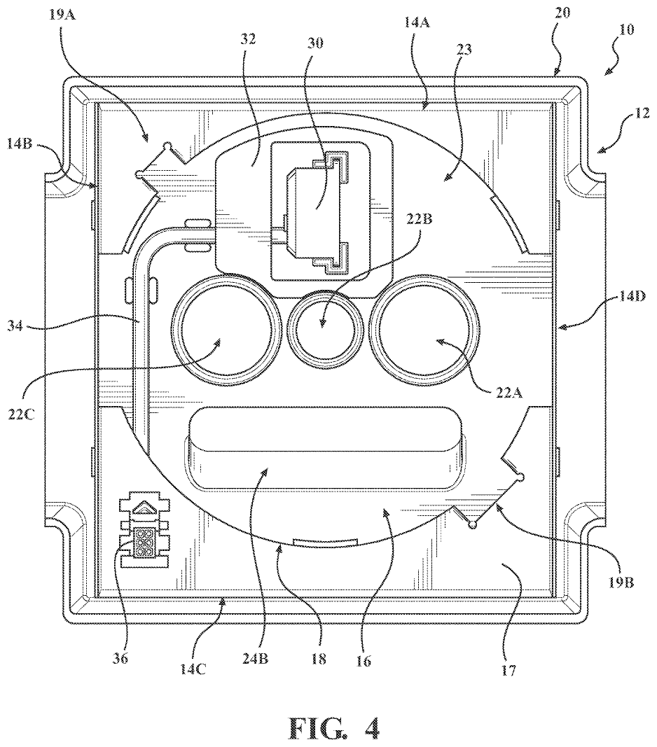

The housing 12 may further comprise a cover 20. The cover 20 may be configured to be removably coupled to a distal end of the housing 12. It is also contemplated that the cover 20 may be integrally formed with the wall member(s) 14A, 14B, 14C, 14D. The cover 20 may comprise one or more sockets 22. The aperture may also be referred to as an opening, aperture, outlet, plug, connector, port, or similar means of defining a connection point For example, as illustrated in FIGS. 1 and 2, the cover 20 may comprise three sockets 22A, 22B, 22C. However, it is contemplated that the cover 20 may be configured to comprise a single socket 22A, two sockets 22A, 22B, or more. The cover 20 may also define a recess 24A in the outer surface 21 of the cover 20. The design of the cover 20 may be configured such that the recess 24A of the outer surface 21 of the cover 20 may define a protrusion 24B on the inner surface 23 of the cover 20 (protrusion 24B illustrated in FIG. 4). The recess 24A defined in the outer surface 21 of the cover 20 may be shaped and/or configured as a handle for grabbing and/or manipulating the filter assembly 10. For example, the recess 24A may be configured as a handle to allow the user to grab the filter assembly 10 when removing it from the medical waste collection system 100 or medical device.

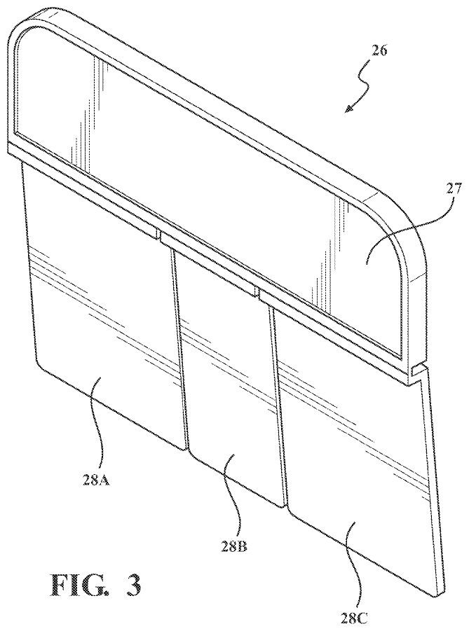

Referring to FIG. 3, an exemplary configuration of an aperture cover 26 for use with the cover 20 is illustrated. The aperture cover 26 may comprise a body 27 configured to be coupled to the outer surface 21 of the cover 20. The body 27 may be removably coupled to the cover 20. Alternatively, the body 27 may be permanently coupled to the cover 20. The body 27 may be formed from a flexible material such as a rubber or from a generally rigid material such as a plastic. The aperture cover 26 may comprise one or more flaps 28 extending from the body 27. For example, as illustrated in FIG. 3, the aperture cover 26 comprises three flaps 28A, 28B, 28C extending from the body 27. The flaps 28A, 28B, 28C may be formed from a flexible material, such as a rubber composition. Furthermore, the flaps 28A, 28B, 28C may be flexibly or pivotably connected to the body 27. For example, the flaps 28A, 28B, 28C may be pivotably connected to the body 27 so that the flaps 28A, 28B, 28C may be moved relative to the body 27 between an open position and a closed position. The body 27 of the aperture cover 26 may be coupled to the cover 20 of the housing 12 such that the flaps 28A, 28B, 28C are positioned over the respective aperture(s) 22A, 22B, 22C of the cover 20. The flaps 28A, 28B, 28C may be configured to cover and/or seal the respective socket 22A, 22B, 22C when in the closed position. The flaps 28A, 28B, 28C may also be configured to expose the respective socket 22A, 22B, 22C when in the open position. Functionally, when the flaps 28A, 28B, 28C are in the closed position and covering the respective socket 22A, 22B, 22C, the flaps 28A, 28B, 28C may prevent debris or other particles from passing through the aperture(s) 22A, 22B, 22C of the cover 20. When the filter assembly 10, including a cover 20 with the aperture cover 26, is utilized with a medical waste collection system 100 including a vacuum, the vacuum may cause the flaps 28A, 28B, 28C to seal against the outer surface of the cover 20 when the flaps 28A, 28B, 28C are in the closed position. Alternatively, when the flaps 28A, 28B, 28C are in the open position, the filter assembly 10 may be configured such that the vacuum may draw liquid, gases, and/or other particles through the sockets 22A, 22B, 22C of the cover 20.

Referring to FIG. 4, a rear view of the housing 12 of the filter assembly 10 is illustrated. The proximal end of the housing 12 may define an opening 18 configured to provide access to the interior 16 defined by the wall member(s) 14A, 14B, 14C, 14D. While the opening 18 in the proximal end of the housing 12 illustrated in FIG. 4 comprises a generally circular shape, it is contemplated that other shapes may be utilized. For example, the opening 18 may comprise a triangular shape, a square shape, or other similar polygonal shape.

The opening 18 in the proximal end of the housing 12 may also define one or more alignment slots 19A, 19B. The alignment slots 19A, 19B may comprise a generally square or rectangular cut-out in the perimeter of the opening 18. However, it is further contemplated that the alignment slots 19A, 19B may comprise a hemispherical concave shape, a triangular shape, or other polygonal cut-out. For example, as illustrated in FIG. 4, the opening 18 comprises a first alignment slot 19A and a second alignment slot 19B. The first alignment slot 19A comprises a generally rectangular shape having a first dimension and the second alignment slot 19B comprises a generally rectangular shape having a second dimension, wherein the first dimension and the second dimension are distinct from one another. As illustrated in FIG. 4, the exemplary configuration of the opening 18 includes a first alignment slot 19A comprising a first dimension and a second alignment slot 19B comprising a second dimension, wherein the first dimension is smaller than the second dimension. Alternatively, while not illustrated in the figures, it is also contemplated that the alignment slots 19A, 19B may be configured as an alignment tab protruding from the perimeter of the opening 18. For example, the alignment tab may comprise a hemispherical convex shape, a triangular shape, or similar polygonal shape extending from the perimeter of the opening 18. Furthermore, while the figures only show the alignment slots 19A, 19B cut out of the perimeter of the opening 18 in proximal panel 17 of the housing 12, it is contemplated that the interior 16 defined by the wall members 14A, 14B, 14C, 14D may comprise the same shape as the opening 18. For example, the interior 16 defined by wall members 14A, 14B, 14C, 14D may comprise a cylindrical shape extending from the proximal end to the proximal end of the housing 12, wherein the interior surface of the cylinder may define one or more alignment slots 19A, 19B that extend the length of the cylinder. While the alignment slots 19A, 19B illustrated in FIG. 4 generally bisect the opening 18, other arrangements and/or orientations are contemplated. For example, visualizing the opening 18 as a clock face, it is contemplated that the first alignment slot 19A may be positioned at approximately eleven o'clock (as illustrated in FIG. 4), and the second alignment slot 19B may be positioned at approximately one o'clock. In yet another configuration, the first alignment slot 19A may be positioned at approximately one o'clock, and the second alignment slot 19B may be positioned at approximately five o'clock (as illustrated in FIG. 4).

The filter assembly 10 may further comprise a particulate sensor 30 disposed within the interior 16 of the housing 12. The particulate sensor 30 may comprise an optical sensor, a chemical sensor, or a laser sensor. For example, an optical particulate sensor 30 may comprise an infrared emitting diode (IRED) and an phototransistor are diagonally arranged such that the optical particulate sensor 30 may detect the reflected light of dust in air. This type of optical particulate sensor 30 may generally be effective to detect very fine particle such as cigarette smoke. The particulate sensor 30 may comprise a sensor housing 31 and a sensor element 33. As illustrated in FIG. 4, the sensor housing 31, and by extension the sensor 30, may be coupled to or mounted on the cover 20 of the housing 12. While not illustrated in the figures, it is further contemplated that the particulate sensor 30 may be mounted at other locations within the interior 16 of the housing 12. For example, the particulate sensor 30 may be mounted to the first wall member 14A. Alternatively, the particulate sensor 30 may be mounted to the second wall member 14B. The sensor element 33 may comprise the operative feature of the sensor, such as an optical bulb of an optical sensor configured to visually detect the presence of various particulates. Alternatively, the sensor element 33 may comprise a chemical analyzer configured to detect the presence of various particles. Extending from the sensor housing 31 of the particulate sensor 30 is a sensor cord 34. The sensor cord 34 may comprise a sensor fitting 36 coupled to the sensor cord 34 opposite the sensor 30. The sensor fitting 36 may be configured to be coupled to a complementary fitting on the medical waste collection system 100 or medical device to connect the particulate sensor 30 to a controller 102 of the system 100 or device.

The filter assembly 10 may further comprise a seal member 32 disposed within the interior 16 of the housing 12. The seal member 32 may be coupled to the cover 20 and positioned to at least partially surround the sensor 30. A portion of the seal member 32 may also at least partially encircle a segment of the sensor cord 34 to create a seal between the sensor cord 34 and the cover 20 of the housing 12.

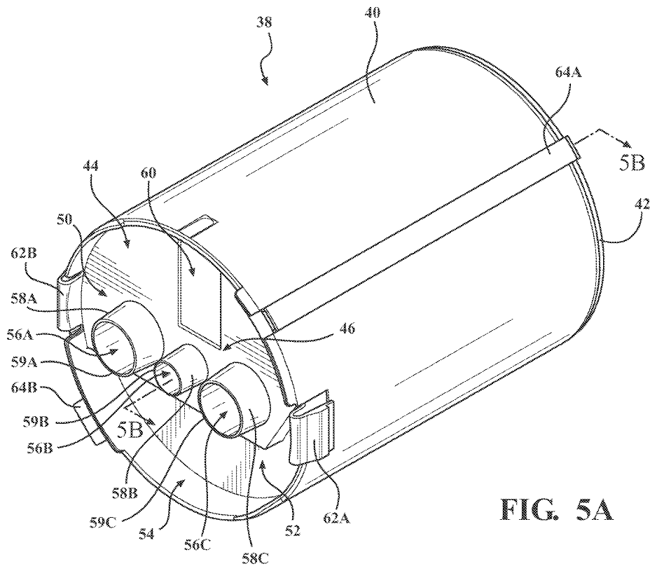

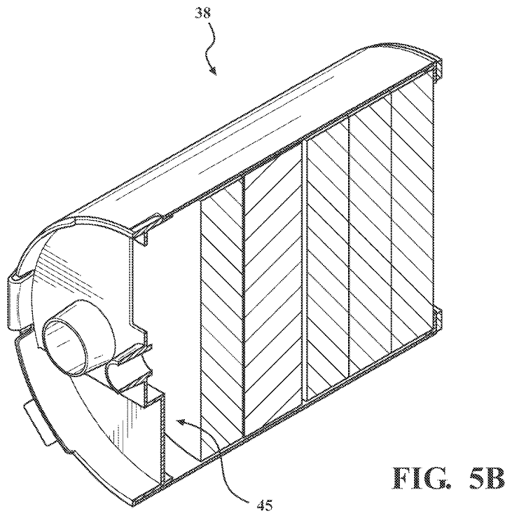

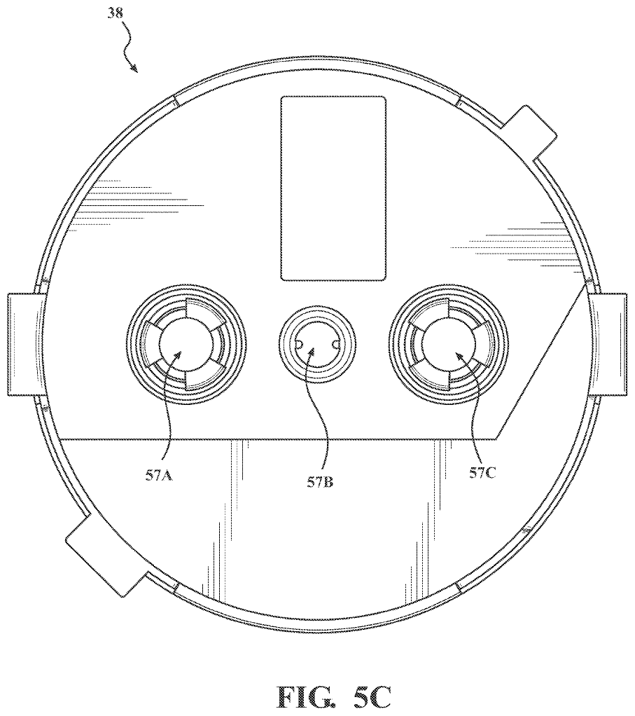

Referring to FIGS. 5A, 5B, and 5C, an exemplary configuration of a filter cartridge 38 of the filter assembly 10 is illustrated. The filter cartridge 38 may be configured to be removably disposed within the interior 16 of the housing 12. The filter cartridge 38 may comprise a cartridge cover 40 configured to define an outer perimeter of the filter cartridge 38. As illustrated in FIG. 5A, the cartridge cover 40 may comprise a generally cylindrical shape. However, it is also contemplated that the cartridge cover 40, and by extension the filter cartridge 38, may be configured in alternative shapes. For example, the cartridge cover 40 may be configured as a square cuboid, rectangular cuboid, or similar three-dimensional shape. The shape of the filter cover 40, and by extension the filter cartridge 38, may generally be configured to allow the filter cartridge 38 to be inserted through the opening 18 in the proximal end of the housing 12.

The filter cartridge 38 may also be coated with a liquid impermeable coating 41 configured to prevent liquid from entering the filter cartridge 38. The liquid impermeable coating 67 may be disposed on one of an exterior surface or an interior surface of the filter cartridge 38, such as the interior or exterior surface of the cartridge cover 40. It is also contemplated that the liquid impermeable coating 67 may be disposed on both the exterior surface and the interior surface of the filter cartridge 38, such as the interior and exterior surface of the cartridge cover 40. The liquid impermeable coating 41 may be configured to prevent liquid from entering the filter cartridge 38 and potentially damaging internal components of the filter cartridge 38, such as the filter portion 66, which will be discussed in greater detail below. It is also contemplated that the liquid impermeable coating 41 may also prevent liquids from leaking through or exiting from the filter cartridge 38. For example, prevent liquids from leaking through the filter cartridge 38 and potentially contaminating the interior 16 of the housing 12.

It is further contemplated that at least one of the interior of the smoke evacuation tube 110 and/or the interior chamber of the filter cartridge 38 may comprise an absorbent material configured to prevent damage to the filter cartridge 38 and or the internal components of the filter cartridge 38. For example, the absorbent material may be a Aqueous Superabsorbent Coating (ASC) technology that is a liquid polymer solution that dries to form an absorbent film. The ASC may be applied using various techniques to a range of materials and substrates to absorb condensed or evaporated fluids. ASC is delivered as a liquid suspension that when dried, self-crosslinks to form a superabsorbent film coating that can absorb many times its own weight of water. Water vapor, bodily fluids, and other aqueous solutions may also be absorbed by the coating.

The filter cartridge 38 may further comprise one or more alignment features 64A, 64B positioned on the exterior of the cartridge cover 40. As illustrated in FIG. 5A, the alignment feature(s) 64A, 64B may be configured as a rail, tab, or protrusion extending from the exterior of the cartridge cover 40. The alignment feature(s) 64A, 64B may be positioned on the exterior of the cartridge cover 40 to align and/or orient the filter cartridge 38 within the housing 12. For example, as illustrated in FIG. 5A, the alignment features 64A, 64B may comprise a rail that extends the length of the filter cartridge 38. Alternatively, the alignment features 64A, 64B may comprise a protrusion or tab positioned at a point along the filter cartridge 38. For example, the alignment features 64A, 64B may comprise a protrusion positioned at the distal end of the filter cartridge 38. The alignment feature(s) 64A, 64B may be configured to be at least partially disposed within the complementary alignment slot 19A, 19B defined in the opening 18 of the housing 12 when the filter cartridge 38 is disposed in the housing 12. For example, the first alignment feature 64A may be aligned with and at least partially disposed within the first alignment slot 19A in the opening 18, and the second alignment feature 64B may be aligned with and at least partially disposed within the second alignment slot 19B in the opening 18. Alternatively, as described above, when the opening 18 comprises an alignment tab, the cartridge cover 40 may be configured to comprise an alignment groove that is configured to at least partially receive the alignment tab(s) of the opening 18 to align and/or orient the filter cartridge 38 within the housing 12.

The filter cartridge 38 may further comprise a rear plate 42 and a faceplate 44 coupled to opposing ends of the cartridge cover 40. The rear plate 42 may be positioned at the proximal end of the filter cartridge 38 and the faceplate 44 may be positioned at the distal end of the filter cartridge 38. The rear plate 42 and the faceplate 44 may be coupled to the cartridge cover 40 to define an interior chamber 45 of the filter cartridge 38.

The faceplate 44 of the filter cartridge 38 may comprise an outer surface 46 and an opposing inner surface 48, with a rim 54 encircling an outer perimeter of the faceplate 44. The outer surface 46 may comprise a first portion 50 and a second portion 52, wherein the second portion 52 is positioned proximal to the first portion 50 to define a recess in the outer surface 46. The recess defined by the second portion 52 of the outer surface 46 may be configured to receive the protrusion 24B on the inner surface 23 of the cover 20 when the filter cartridge 38 is disposed within the interior 16 of the housing 12.

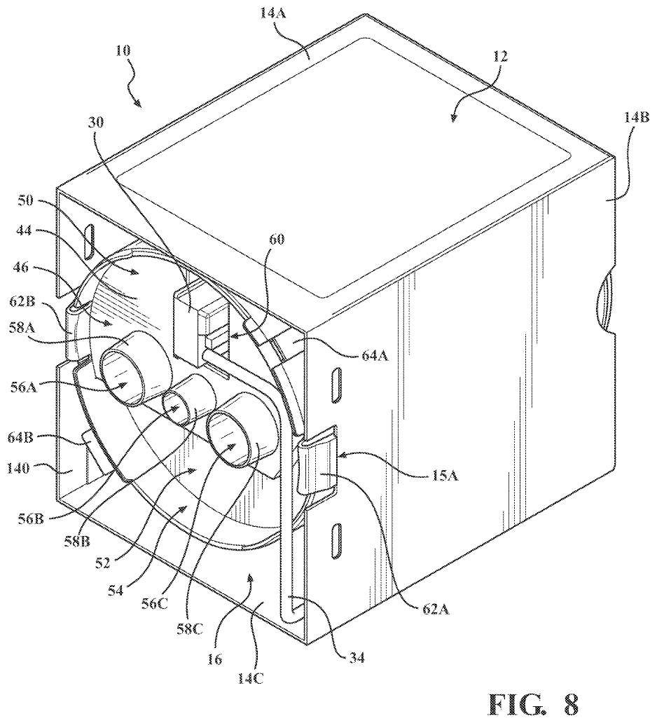

The rim 54 encircling the outer perimeter of the faceplate 44 may project distally from the outer surface 46. The rim 54 may also project proximally from the inner surface 48 of the faceplate 44. The rim 54 may also comprise one or more coupling members 62A, 62B configured to removably couple the filter cartridge 38 to the housing 12 when disposed within the interior 16. As illustrated in FIG. 5A, the coupling members 62A, 62B may comprise tabs that form a portion of the rim 54 and are positioned on opposing sides of the faceplate 44. However, it is also contemplated that the coupling members 62A, 62B may positioned at alternative locations on the faceplate 44 and/or the filter cartridge 38. For example, the coupling members 62A, 62B may be coupled to or formed as part of the cartridge cover 40. As will be described in greater detail below, the wall members 14A, 14B, 14C, 14D of the housing 12 may comprise a complementary coupling feature 15A, 15B, such as a cut-out or opening, configured to engage the coupling members 62A, 62B of the filter cartridge 38 (see FIG. 8).

The faceplate 44 of the filter cartridge 38 may further comprise one or more openings defining ports 56A, 56B, 56C in the faceplate 44. As illustrated in FIG. 5A, the faceplate 44 may comprise three ports 56A, 56B, 56C that may be arranged in a generally linear configuration and be positioned on the first portion 50 of the outer surface 46 of the faceplate 44. However, other configurations and arrangements of the ports 56A, 56B, 56C are contemplated. For example, the faceplate 44 may comprise only a single port 56A, or it may comprise two ports 56A, 56B. It is also contemplated that the faceplate 44 may comprise more than three ports 56A, 56B, 56C . . . 56n. While not illustrated in the figures, it is also contemplated that the ports 56A, 56B, 56C may be positioned at alternative locations on the outer surface 46 of the faceplate 44, such as the open space on the faceplate 44 above the first port 56A. Each of the ports 56A, 56B, 56C may be configured to receive an end or portion of a tube 110, such as a smoke evacuation tube, that is utilized as part of the medical waste collection system 100 or medical device. The end may comprise a smoke evacuation tube coupler or fitting configured to removably couple with the ports 56A, 56B, 56C of the faceplate 44.

Each of the ports 56A, 56B, 56C may also comprise a valve 57A, 57B, 57C disposed within the opening defining the port(s) 56A, 56B, 56C. The valve(s) 57A, 57B, 57C may be configured to control the flow of fluids and/or gases into and out of the filter cartridge 38. For example, the valve(s) 57A, 57B, 57C may comprise a one-way valve that only allows fluids and/or gases to flow into the filter cartridge 38. Alternatively, the valve(s) 57A, 57B, 57C may comprise a two-way valve that allows fluids and/or gases to flow both into and out of the filter cartridge 38. One example of a one-way fluid valve for use with the filter cartridge 38 is a rubber sealing valve, such as a flapper valve, umbrella valve and/or duckbill valve. A one-way rubber sealing valve may be disposed within the port(s) 56A, 56B, 56C and may be configured such that the valve(s) 57A, 57B, 57C opens when an end or portion of a tube 110, such as a smoke evacuation tube, is coupled to the port(s) 56A, 56B, 56C, to allow the flow of fluids and/or gases into the filter cartridge 38. The valve(s) 57A, 57B, 57C may then close when the tube 110 is removed from the port(s) 56A, 56B, 56C, to prevent the flow of fluids and/or gases out of the filter cartridge 38.

Each of the ports 56A, 56B, 56C may further comprise an annular boss 58A, 58B, 58C encircling one of the ports 56A, 56B, 56C and projecting distally from the outer surface 46 of the faceplate 44. The annular bosses 58A, 58B, 58C may be configured to couple the end of the tube 110 to the faceplate 44. Each of the ports 56A, 56B, 56C, and by extension each of the corresponding annular bosses 58A, 58B, 58C, may comprise a different size opening to allow for different size tubes 110 to be coupled to the faceplate 44. For example, each of the annular bosses 58A, 58B, 58C may be sized and configured to receive a tube 110, wherein the annular bosses 58A, 58B, 58C create a friction fit with the tube 110 to couple the tube 110 to the faceplate 44. Alternatively, the annular bosses 58A, 58B, 58C may comprise a fitting or coupling feature, such as a notch or cut-out, and the tube 110 may comprise a complementary fitting including a tab or protrusion configured to engage the coupling feature of the annular bosses 58A, 58B, 58C to removably couple a fitting of the tube 110 to the faceplate 44.

Furthermore, each of the annular bosses 58A, 58B, 58C may comprise a distal end 59A, 59B, 59C. The distal ends 59A, 59B, 59C of the annular bosses 58A, 58B, 58C may be sized to fit within one of the complementary sockets 22A, 22B, 22C of the cover 20 when the filter cartridge 38 is disposed within the interior 16 of the housing 12.

The faceplate 44 of the filter cartridge 38 may further comprise a sensor opening 60. The sensor opening 60 may be positioned on the faceplate 44 such that it receives at least a portion of the particulate sensor 30 when the filter cartridge 38 is disposed within the interior 16 of the housing 12. For example, the sensor opening 60 on the faceplate 44 is configured to receive the particulate sensor 30 so that the particulate sensor 30 is at least partially disposed within the interior chamber 45 of the filter cartridge 38 when disposed within the housing 12. The particulate sensor 30 may be oriented so that the sensor element 33 may directed toward the ports 56A, 56B, 56C of the faceplate 44 when the particulate sensor 30s at least partially disposed within the interior chamber 45 of the filter cartridge 38. This may improve the ability of the particulate sensor 30 to detect the presences of particulates entering the cartridge through the ports 56A, 56B, 56C of the faceplate 44. Furthermore, the seal member 32 that surrounds at least a portion of the particulate sensor 30 may be configured to provide a seal between the outer surface 46 of the faceplate 44 and the inner surface 23 of the cover 20 that surrounds the sensor opening 60 to prevent liquids, gases, or other particulates from exiting the interior chamber 45 of the filter cartridge 38 through the sensor opening 60 when the filter cartridge 38 is disposed within the housing 12. While the seal member 32 is previously described as being coupled to the cover 20, it is also contemplated that the seal member 32 may be coupled to the outer surface 46 of the faceplate 44.

The arrangement and position of the various features of the filter cartridge 38, and more specifically the faceplate 44, may assist in orienting the filter cartridge 38 within the housing 12. As described above, the position of the alignment slot(s) 19A, 19B of the housing 12 and the alignment feature(s) 64A, 64B may be complementarily arranged to align the ports 56A, 56B, 56C of the faceplate 44 with the aperture(s) 26A, 26B, 26C of the cover 20 when the filter cartridge 38 is disposed within the housing 12. The position of the alignment slot(s) 19A, 19B of the housing 12 may similarly orient the sensor opening 60 relative to the sensor 30, such that the particulate sensor 30 may be at least partially disposed within the sensor opening 60 when the filter cartridge 38 is disposed within the housing 12. For example, as described above the first alignment slot 19A may be positioned at approximately eleven o'clock and the second alignment slot 19B may be positioned at approximately five o'clock. The complementary alignment features 64A, 64B may be positioned at the same location on the filter cartridge 38. The particulate sensor 30 may then be positioned at approximately twelve o'clock on the cover 20 and the sensor opening 60 may similar be positioned at approximately twelve o'clock. This arrangement of the complementary features allows the particulate sensor 30 to be at least partially disposed within the sensor opening 60 when the filter cartridge 38 is disposed within the housing 12.

The position of the alignment feature(s) 64A, 64B of the filter cartridge 38 may be complementarily arranged relative to the position the annular boss(es) 58A, 58B, 58C of the faceplate 44 so that the annular boss(es) 58A, 58B, 58C are disposed within aperture(s) 26A, 26B, 26C of the cover 20 when the filter cartridge 38 is disposed within the housing 12. For example, as described above the first alignment feature 64A may be positioned at approximately eleven o'clock and the second alignment feature 64B may be positioned at approximately five o'clock. The annular bosses 58A, 58B, 58C may be arranged in a generally linear configuration along an axis extending between three o'clock and nine o'clock. The complementary apertures 26A, 26B, 26C in the cover 20 may be similarly arranged such that the annular boss(es) 58A, 58B, 58C are disposed within aperture(s) 26A, 26B, 26C of the cover 20 when the filter cartridge 38 is disposed within the housing 12. The combination of these various complementary features insure the filter cartridge 38 is inserted within the housing 12 in a single orientation so that the particulate sensor 30 may be disposed within the senor opening 60 and the annular bosses 58A, 58B, 58C may be disposed within the apertures 26A, 26B, 26C when the filter cartridge 38 is disposed within the housing 12. While not described I detail, other configurations or arrangements of the ports 56A, 56B, 56C, relative to the aperture(s) 26A, 26B, 26C, and the sensor opening 60 relative to the particulate sensor 30 are contemplated.

Referring to FIG. 5B, a sectional view of the filter cartridge 38 is illustrated to show various internal components of the filter cartridge 38. The filter cartridge 38 of the filter assembly 10 may further comprise a filter portion 66 disposed within the interior chamber 45 defined by the cartridge cover 40. The filter portion 66 of the filter cartridge 38 may comprise one or more filter layers 68A, 68B, 68C, 68D, 68E. Each of filter layers 68A, 68B, 68C, 68D, 68E may comprise differing materials and/or pore sizes configured to collect different sizes and/or types of particulates as liquids or gases pass through the various filter layers 68A, 68B, 68C, 68D, 68E. For example, one or more of the filter layers 68A, 68B, 68C, 68D, 68E may comprise a fibrous or porous materials which remove solid particulates such as dust, pollen, mold, and bacteria from the air. Alternatively, one or more of the filter layers 68A, 68B, 68C, 68D, 68E may comprise an adsorbent or catalyst such as charcoal (carbon) that may also remove odors and/or gaseous pollutants such as volatile organic compounds or ozone. For example, a first filer layer 68A may comprise a thick tilter foam including a melt-blown plastic material configured to capture bigger particle before ULPA filter media. A second filer layer 68B may comprise a ULPA pleat pack including a filter media that has approximately 40 pleats and is 1.5 inches deep. It may be used with a cardboard tube and glued with spin glue technology. Additional layers 68C,68D,68E of the filter portion 66 may comprise activated carbon such as activated carbon foam of 5.0″ ID used and glued with cardboard tube.

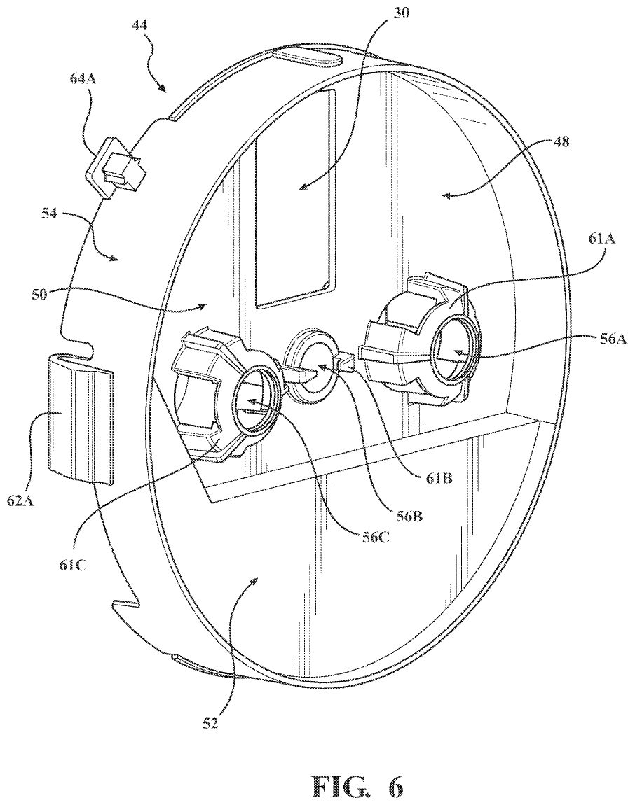

Referring to FIG. 6, a perspective view of the inner surface 48 of the faceplate 44 is illustrated. The faceplate 44 of the filter cartridge 38 may further comprise one or more cages 61A, 61B, 61C positioned on and projected distally from the inner surface 48 of the faceplate 44. Each of the cages 61A, 61B, 61C may be configured to at least partially encircle the ports 56A, 56B, 56C of the faceplate 44. The cage(s) 61A, 61B, 61C may be configured to prevent over-insertion of a tube 110 coupled to the faceplate 44 via the ports 56A, 56B, 56C and/or the annular bosses 58A, 58B, 58C. For example, a tube 110 may be slid into the annular bosses 58A, 58B, 58C and through the ports 56A, 56B, 56C in the faceplate 44. The cage(s) 61A, 61B, 61C may be positioned on the inner surface 48 of the faceplate 44 and configured to stop the fitting of the tube 110 once it has passed through the faceplate 44.

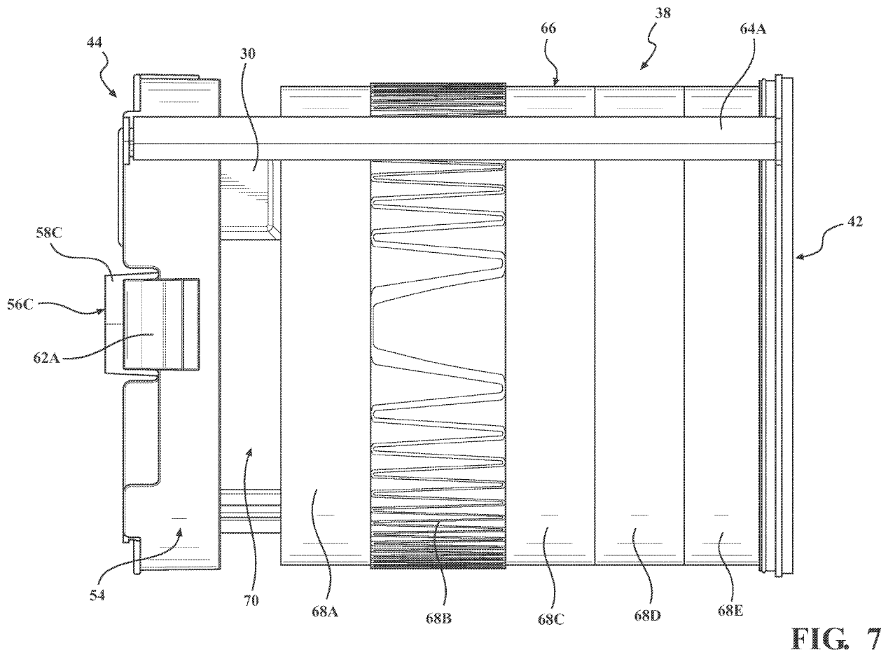

Referring to FIG. 7, the filter cartridge 38 is illustrated without the cartridge cover 40 is illustrated. While the housing 12 is not illustrated in FIG. 7, the particulate sensor 30 is illustrated within the interior chamber 45 of the filter cartridge 38 as if the filter cartridge 38 were disposed within the housing 12. As can be seen in FIG. 7, the filter portion 66 of the filter cartridge 38 may be spaced apart from the faceplate 44, and more specifically spaced apart from the inner surface 48 (not visible in FIG. 7) of the faceplate 44. The filter portion 66 may be spaced apart from the inner surface 48 of the faceplate 44 to define a void space 70 within the interior chamber 45 of the filter cartridge 38. The ports 56A, 56B, 56C and the sensor opening 60 may be in fluid communication with the void space 70. The void space 70 may be configured to receive smoke, liquid, gases, or other particulates collected via the tube(s) 110 that are coupled to the ports 56A, 56B, 56C of the faceplate 44. The void space 70 may be configured to receive at least a portion of the particulate sensor 30 via the sensor opening 60, such that at least a portion of the particulate sensor 30 is disposed within the void space 70 when the filter cartridge 38 is disposed within the housing 12. The particulate sensor 30 may be configured to detect the presence of smoke, liquid, gases, or other particulates entering the filter cartridge 38 via the ports 56A, 56B, 56C. For example, as described above, the particulate sensor 30 may comprise an optical sensor configured to detect the presence of smoke within the void space 70. Alternatively, it is further contemplated that the particulate sensor 30 may comprise a chemical sensor configured to detect the presence of gases or particulates other than oxygen within the void space 70.

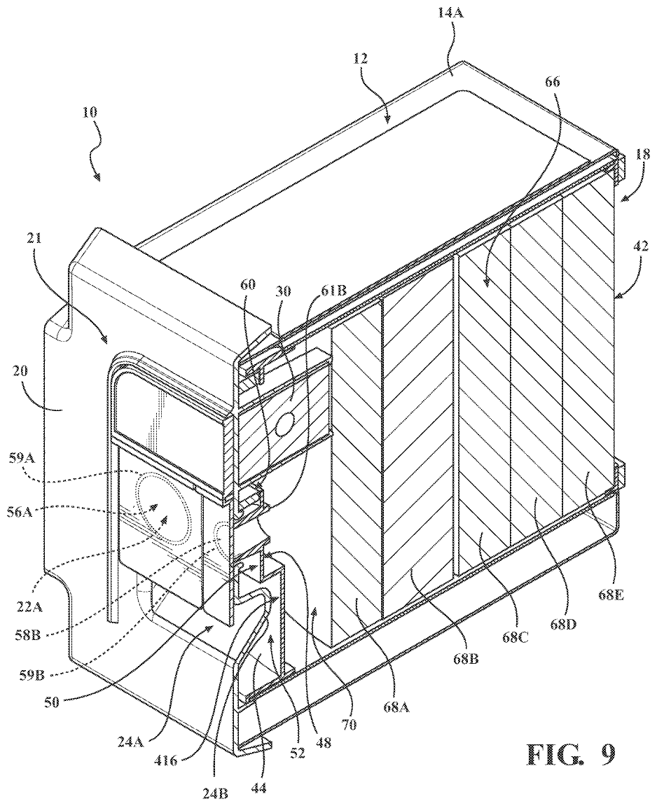

Referring to FIGS. 8 and 9, various views of the filter cartridge 38 disposed within the housing 12 are illustrated. FIG. 8 provides a perspective view of the filter cartridge 38 disposed within the housing 12. The cover 20 of the housing 12 is omitted to show the positioning and relationship of the filter cartridge 38 and the housing 12. Specifically, as can be seen in FIG. 8, the particulate sensor 30 may be inserted through the sensor opening 60 so that the particulate sensor 30 may be at least partially disposed within the filter cartridge 38. Furthermore, an exemplary configuration of the coupling members 62A, 62B of the filter cartridge 38 and the complementary coupling features 15A, 15B of the wall members 14A, 14B, 14C, 14D of the housing 12 are illustrated. For example, the coupling members 62A, 62B of the filter cartridge 38 may comprise a tab that is configured to engage the complementary coupling features 15A, 15B that comprise a notch in the wall members 14A, 14C. The coupling members 62A, 62B may snap into the complementary coupling features 15A, 15B when the filter cartridge 38 is inserted into the housing 12. Alternatively, the coupling members 62A, 62B may be manipulated or depressed to disengage them from the complementary coupling features 15A, 15B to remove the filter cartridge 38 from the housing 12.

FIG. 9 provides a sectional view of the filter assembly 10 including the filter cartridge 38 disposed within the housing 12. Specifically, FIG. 9 illustrates an exemplary configuration of the filter assembly 10 wherein the filter cartridge 38 is disposed within the housing 12 and how the faceplate 44 interacts with the cover 20 of the housing 12. For example, as seen in FIG. 9, the annular boss(es) 58A, 58B, 58C of the faceplate 44 may be at least partially disposed within the sockets 22A, 22B, 22C of the cover 20. It is contemplated that the distal end(s) 59A, 59B, 59C of the annular boss(es) 58A, 58B, 58C may be positioned such that they are flush with the outer surface 21 of the cover 20 when the filter cartridge 38 is disposed within the interior 16 of the housing 12. Alternatively, it is also contemplated that the distal end(s) 59A, 59B, 59C of the annular boss(es) 58A, 58B, 58C may be positioned such that they are distal to and/or extend beyond the outer surface 21 of the cover 20 when the filter cartridge 38 is disposed within the interior 16 of the housing 12. Positioning the distal end(s) 59A, 59B, 59C of the annular boss(es) 58A, 58B, 58C either flush with or distal to the outer surface 21 of the cover 20 allows the smoke evacuation tube 110 to be coupled directly to the filter cartridge 38. This greatly reduces the likelihood that any particulates collected through the smoke evacuation tube 110 will end up within the filter cartridge 38 and not the housing 12. This reduces the likelihood of contamination of and/or the need to sterilize the housing 12 between medical procedures. Further still, it is contemplated that the distal end(s) 59A, 59B, 59C of the annular boss(es) 58A, 58B, 58C may be positioned such that they are proximal to the outer surface 21 of the cover 20 when the filter cartridge 38 is disposed within the interior 16 of the housing 12. While this configuration may still allow the smoke evacuation tube 110 to be coupled directly to the filter cartridge 38, it may be less desirable for coupling the smoke evacuation tube 110 to the port(s) 56A, 56B, 56C and may be more likely to result in contamination of the housing 12.

FIG. 9 also illustrates an exemplary configuration of the filter assembly 10 wherein the protrusion 24B on the inner surface 23 of the cover 20 is at least partially disposed within the recess defined by the first portion 50 and the second portion 52 of the faceplate 44. For example, as shown in FIG. 9, the protrusion 24B on the inner surface 23 of the cover 20 defined by the recess in the outer surface 21 of the cover 20 is positioned adjacent the second portion 52 of the faceplate 44 when the filter cartridge 38 is disposed within the interior 16 of the housing 12.

While not illustrated in FIG. 9, it should be understood that the aperture cover 26 may be coupled to the outer surface 21 of the cover 20. As described above, the flaps 28A, 28B, 28C may be pivotably connected to the body 27 of the aperture cover 26 so that the flaps 28A, 28B, 28C may be moved relative to the body 27 between an open position and a closed position. The flaps 28A, 28B, 28C may be configured to cover and/or seal the respective sockets 22A, 22B, 22C and/or the ports 56A, 56B, 56C when in the closed position. The flaps 28A, 28B, 28C may also be configured to expose the respective sockets 22A, 22B, 22C and/or the ports 56A, 56B, 56C when in the open position.

In operation, the filter assembly 10, including the filter cartridge 38 disposed in the housing 12, may be installed in or coupled to the medical waste collection system 100. This process will be described in greater detail below. Once the filter assembly 10 is installed within the medical waste collection system 100, a tube 110, such as a smoke tube, may be coupled to the filter assembly 10 by coupling the fitting of the tube 110 to one of the ports 56A, 56B, 56C and/or annular boss(es) 58A, 58B, 58C of the filter cartridge 38. A vacuum may then be applied to the proximal end of the filter cartridge 38 by a vacuum source of the medical waste collection system 100. For example, when the filter assembly 10 is installed in the medical waste collection system 100, the rear plate 42 of the filter cartridge 38 may be coupled to a vacuum source of the medical waste collection system 100 that is configured to draw a vacuum though the filter cartridge 38. The vacuum applied to the filter cartridge 38 may cause a vacuum to be drawn through the tube 110 that is connected to any of the ports 56A, 56B, 56C and/or annular boss(es) 58A, 58B, 58C of the filter cartridge 38. Particulates will be collected via the tube 110 and deposited in the filter cartridge 38. The particulates may include fluids, smoke, or other gases. The particulates will initially accumulate in the void space 70 between the filter portion 66 and the inner surface 48 of the faceplate 44, prior to being drawn toward the filter portion 66. The particulate sensor 30 may be positioned within the void space 70 and configured to detect the presence of the particulates in the void space 70. The particulate sensor 30 may then send a signal to the controller 102 of the medical waste collection system 100. The controller 102 of the medical waste collection system 100 may be configured to adjust the settings of the vacuum source based, at least in part, on the signal received from the sensor 30. For example, when no particulates are detected by the particulate sensor 30 within the void space 70, the controller 102 may be configured to operate the vacuum at a lower power setting. Alternatively, when particulates are detected by the particulate sensor 30 within the void space 70, the controller 102 may be configured to operate the vacuum at a higher power setting to assist with drawing the particulates into and through the filter cartridge 38. The various filter layers 68A, 68B, 68C, 68D, 68E of the filter portion 66 may then filter out the various particulates as they are pulled though the filter portion 66 by the vacuum.

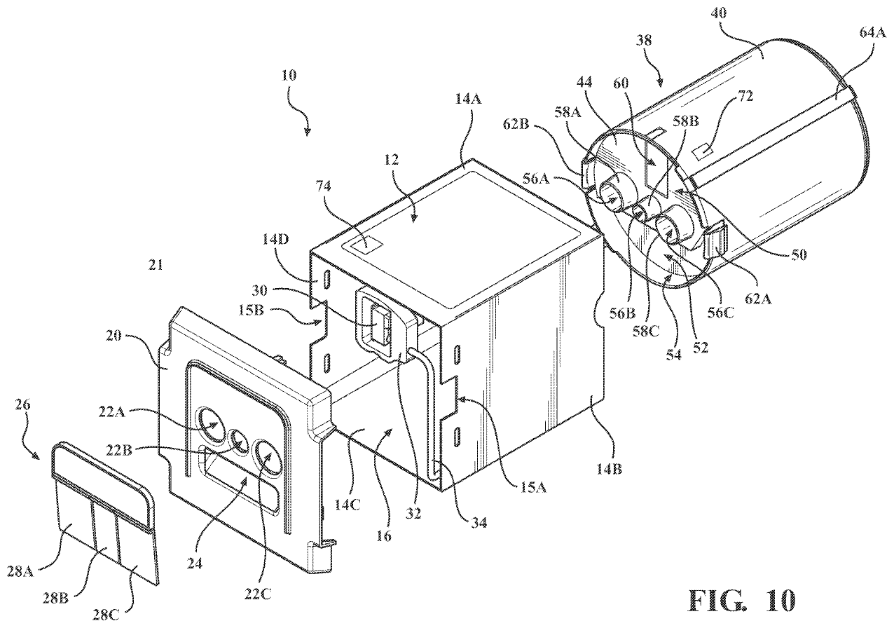

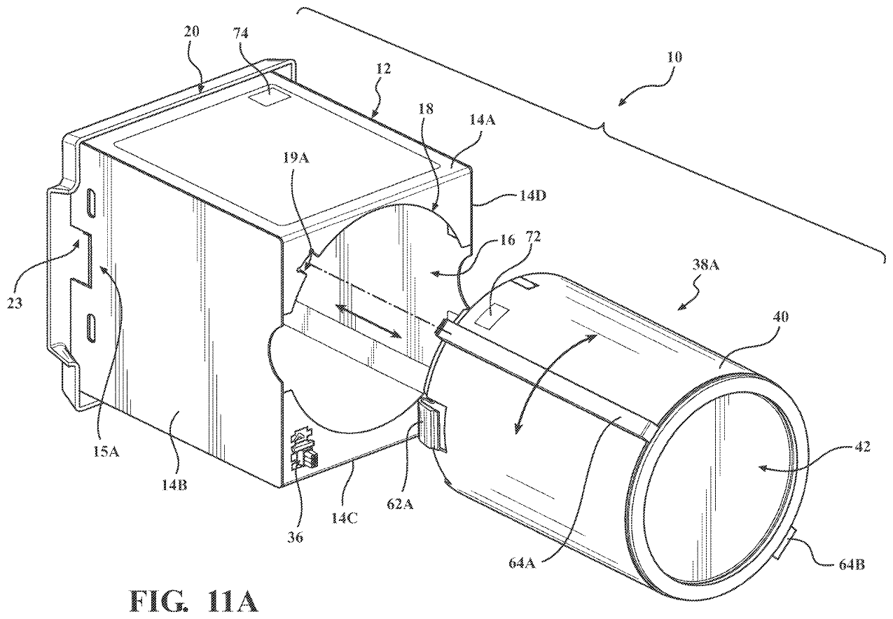

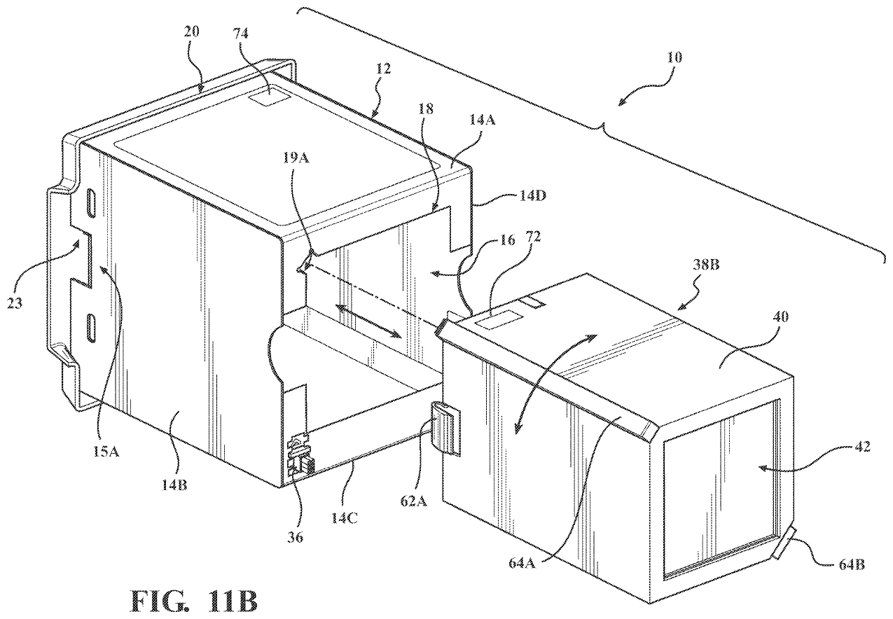

Referring to FIGS. 10 through 11B, various partially exploded views of exemplary configurations of the filter assembly 10 are illustrated. The partially exploded views assist in illustrating various steps of the method of changing the filter assembly 10. Specifically, FIGS. 10 and 11A illustrate various steps in the method of changing the filter assembly 10 related to removing and installing the filter cartridge 38/38A. FIG. 11B illustrates a filter assembly 10 including an alternate filter cartridge 38B. While the filter cartridge 38/38A illustrated in FIGS. 10 and 11A comprises a generally cylindrical shape, as illustrated in FIG. 11B it is also contemplated that the filter cartridge 38B may comprise a generally square cuboid, rectangular cuboid, or similar three-dimensional shape, as described above. The opening 18 in the housing 12 and the slot 19A, 19B may be modified to fit and/or receive the filter cartridge 38, 38A, 38B. This is true for any configuration of the filter cartridges 38, 38A, 38B of the filter assembly 10 described above or below.

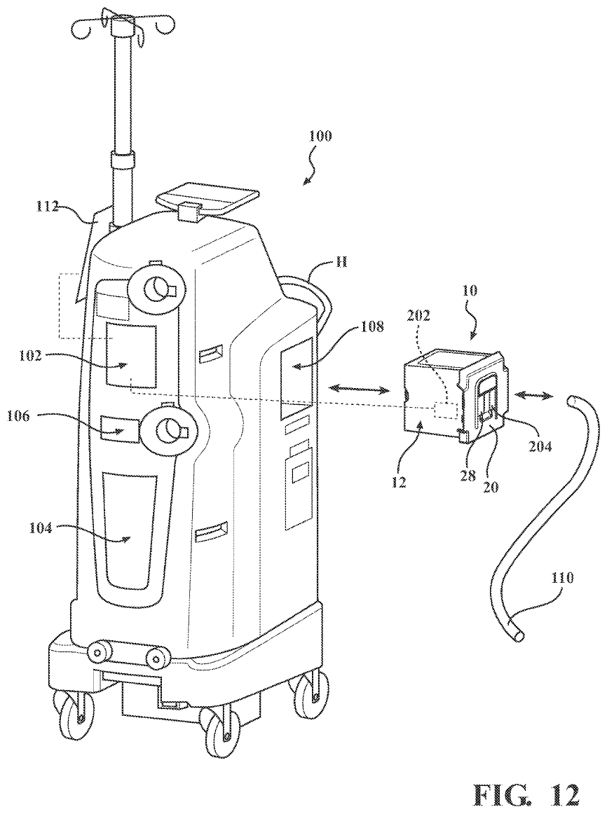

Referring to FIG. 12, a partially exploded view of an exemplary configuration of a medical waste collection system 100 including the filter assembly 10 described above is illustrated. The medical waste collection system 100 may comprise a capital component (H) configured to receive the filter assembly 10. The capital component may also be referred to as a console, cart, station, receiver, or the like. The capital component (H) may define an opening or receiver 108 for receiving the filter assembly 10.

The partially exploded view assists in illustrating various steps of the method of changing the filter assembly 10 of the medical waste collection system 100.

### Method of Replacing the Filter Assembly 10 of a Medical Waste Collection System 100

An exemplary method of replacing a filter cartridge 38 of a filter assembly 10 for use with a medical waste collection system 100 is described below. As described above, the filter assembly 10 may comprise an housing 12 defining an interior 16, an opening 18 in the housing 12, and a slot 19A, 19B. The enclosure may also comprise a front cover 20 having an socket 22A, 22B, 22C, and a particulate sensor 30 disposed within the housing 12. The method of replacing the filter cartridge 38 of the filter assembly 10 may comprise the step of providing the filter cartridge 38. The filter cartridge 38 may comprise a faceplate 44 having a port 56A, 56B, 56C, to removably couple the smoke evacuation tube 110 fitting to the faceplate 44, an alignment feature 64A, 64B coupled to an exterior of the filter cartridge 38, and a sensor opening 60 in the faceplate 44. It is contemplated that the filter cartridge 38 may comprise any combination of the features described above.

The method may further comprise the step of orienting the filter cartridge 38 so the alignment feature 64A, 64B of the filter cartridge 38 may be inserted within the slot 19A, 19B of the housing 12 prior to sliding the filter cartridge 38 through the opening 18. For example, as illustrated by the arrows in FIG. 11, the filter cartridge 38 may be rotated so that the alignment feature 64A, 64B of the filter cartridge 38 are positioned to be inserted within the slot 19A, 19B of the housing 12.

Once the alignment feature 64A, 64B of the filter cartridge 38 has been aligned with the slot 19A, 19B of the housing 12, the method may comprise the step of installing the filter cartridge 38 within the housing 12 by sliding the filter cartridge 38 through the opening 18 so that the alignment feature 64A, 64B is disposed within the slot 19A, 19B. For example, as illustrated by the arrows in FIG. 11, the filter cartridge 38 may be moved distally relative to the housing 12 by sliding the filter cartridge 38 through the opening 18 in the housing 12 and positioning the filter cartridge 38 within the interior of the housing 12. This should position the particulate sensor 30 at least partially within the sensor opening 60 of the filter cartridge 38, and the port(s) 56A, 56B, 56C of the filter cartridge 38 at least partially within the aperture(s) 22A, 22B, 22C in the cover 20.

The method may further comprise the step of installing the filter assembly 10 within an opening or receiver 108 of the medical waste collection system 100. The step of installing the filter assembly 10 may also comprise connecting the particulate sensor 30 to the medical waste collection system 100. For example, as the filter assembly 10 may be installed in the medical waste collection system 100 by inserting the filter assembly 10 in a receiver 108. When the filter assembly 10 is inserted in the receiver 108, the sensor fitting 36 may be coupled to a complementary fitting on the medical waste collection system 100 to connect the particulate sensor 30 to the controller 102 of the system 100.

The method may further comprise the steps of removing the filter assembly 10 from the medical waste collection system 100 and removing the filter cartridge 38 from the interior 16 of the original housing 12. Once the prior filter cartridge 38 has been removed, the method may further comprise orienting a subsequent filter cartridge 38B so that the alignment feature 64A, 64B of the subsequent filter cartridge 38B may be inserted within the groove in at least one of the wall members 14A, 14B, 14C, 14D. The subsequent filter cartridge 38B may then be installed within the original housing 12 by sliding the subsequent filter cartridge 38B through the opening 18 so that the alignment feature 64A, 64B is disposed within the slot 19A, 19B, the particulate sensor 30 is at least partially disposed within the sensor opening 60 of the subsequent filter cartridge 38B, and the port(s) 56A, 56B, 56C of the subsequent filter cartridge 38B are aligned with the aperture(s) 22A, 22B, 22C in the cover 20. The filter assembly 10, including the subsequent filter cartridge 38B, may then be installed within the medical waste collection system 100. The housing 12, including the cover 20, may be configured to be reusable for multiple procedures. For example, the housing 12 and cover 20 may be reused for three medical procedures, four medical procedures, or more. In an exemplary configuration of the housing 12 and cover 20, it is contemplated that the housing 12 and cover 20 may be reused for four separate medical procedures. However, it may be advantageous replace the filter cartridge 38 after each individual medical procedure. Therefore, the filter cartridge 38 may be removed from the housing 12 and replaced with a new filter cartridge 38B after each medical procedure.

The method may further comprise the step of repeating the steps of removing the filter assembly 10 from the medical waste collection system 100, removing the filter cartridge 38, orienting a subsequent filter cartridge 38B, installing the subsequent filter cartridge 38B, and installing the filter assembly 10 within the medical waste collection system 100.

The method may further comprise the step of counting each occurrence of installing the subsequent filter cartridge within the enclosure indicative of the number of uses of the filter assembly 10. This may be accomplished by including a transceiver or identification tag 72, 74, such as an RFID tag, on each of the housing 12 and the filter cartridge 38 (see FIGS. 10 and 11). Each of the transceivers 72, 74 may comprise a memory that may comprise one or more pieces of datum related to the housing 12 and/or the filter cartridge 38. For example, the transceivers 72, 74 may identify the specific type and/or various characteristics of the housing 12 and/or the filter cartridge 38. This may include a specific serial number or identification number associated with each of the housing 12 and/or the filter cartridge 38. The memory of the transceivers 72, 74 may also include the pore size and/or filtration rating for the filter cartridge 38. The transceivers 72, 74 may also identify the type of housing 12 and the filter cartridge 38 for the purpose of confirming a compatible filter cartridge 38 is installed in the housing 12, and by extension that a compatible housing 12 and/or the filter cartridge 38 are installed in the medical waste collection system 100. The medical waste collection system 100 may comprise a reader 106 or antenna that is coupled to the controller 102, wherein the reader 106 is configured to read the transceiver(s) 72, 74 of the housing 12 and/or the filter cartridge 38, and communicate the data received from the transceiver(s) 72, 74 to the controller 102 of the medical waste collection system 100. The transceiver 72, 74 may be read by a reader 106 or antenna of the medical waste collection system 100, and the data may be transferred to the controller 102. The controller 102 may be configured to use the serial number of the housing 12 and/or the filter cartridge 38 to determine if either the housing 12 and/or the filter cartridge 38 has been previously installed in a medical waste collection system 100. The controller 102 may also confirm that the housing 12 and/or the filter cartridge 38 are compatible with the medical waste collection system 100. The controller 102 may then be configured to operate the medical waste collection system 100 based on the data deceived from the transceiver(s) 72, 74. For example, the controller 102 may be configured to prevent operation of the medical waste collection system 100 if the controller 102 identifies that either of the housing 12 and/or the filter cartridge 38 have exceeded a threshold number of uses or is incompatible with the medical waste collection system 100. The controller 102 may also be configured to adjust the vacuum source of the medical waste collection system 100 based on the type and/or pore size of the filter cartridge 38 that is installed.

Alternatively, the filter assembly 10 may comprise a mechanical counter that will be attached to the cover 20, wherein the counter will rotate to a certain angle when a filter cartridge 38 is replaced. The counter may have a dial or rotatable member including numerals or other markings printed on one side that may be visible from outside the filter assembly 38 through a hole, window, or transparent member in the cover 20. After certain number of replacements of filter cartridge 38, the counter will not be rotated, and filter cartridge 38 may be prevented from being inserted within the housing 12.

Utilizing the filter assembly 10 including the transceiver(s) 72, 74 and/or controller 102 described above, the method may further comprise the step of counting each occurrence of installing the filter assembly 10 within the medical waste collection system 100 indicative of the number of uses of the filter assembly 10. This may also include indicating when the number of uses of the filter assembly 10 has reached a threshold value to inform the user that the filter assembly 10 is due to be replaced. This may include indicating when a threshold number of uses has been reached for either of the housing 12 and/or the filter cartridge 38. For example, when the filter cartridge 38 is intended to be a single use filter cartridge 38, if the controller 102 identifies that the filter cartridge 38 has been previously installed and/or used, the controller 102 may prevent operation of the medical waste collection system 100. In another example, when the housing 12 is intended to be a used for a maximum of four procedures, if the controller 102 identifies the housing 12 has been previously installed and/or used for four medical procedures, the controller 102 may prevent operation of the medical waste collection system 100.

The method may further comprise the step of providing a visual indicator of the number of occurrences of installing the subsequent filter cartridge 38 within the housing 12 and/or the number of occurrences of installing the filter assembly 10 within the medical waste collection system 100.

The method may further comprise the step of counting each occurrence of the step of installing the filter cartridge 38 within the housing 12 by sliding the filter cartridge 38 through the opening 18 indicative of the number of uses of the filter assembly 10. While not illustrated, it is contemplated that the housing 12 may comprise an antenna similar to the antenna 106 of the medical waste collection system 100, wherein the antenna of the housing 12 is configured to the read the data from the transceiver 72 of the filter cartridge and communicate the data to the controller 102 of the medical waste collection system 100. The operation of the controller 102 and/or the medical waste collection system 100 may be similar to or the same as described above with regard to operation of the medical waste collection system 100 based on data communicated between the transceiver(s) 72, 74 of the filter assembly 10 and the antenna 106 of the medical waste collection system 100. For example, the controller 102 may be configured to count each occurrence of the step of installing the cartridge 38 within the housing 12 indicative of the number of uses of the filter assembly 10. The controller 102 may then be configured to indicate when the number of uses of the filter assembly 10 has reached a threshold value to inform the user the filter assembly 10 is due to be replaced.

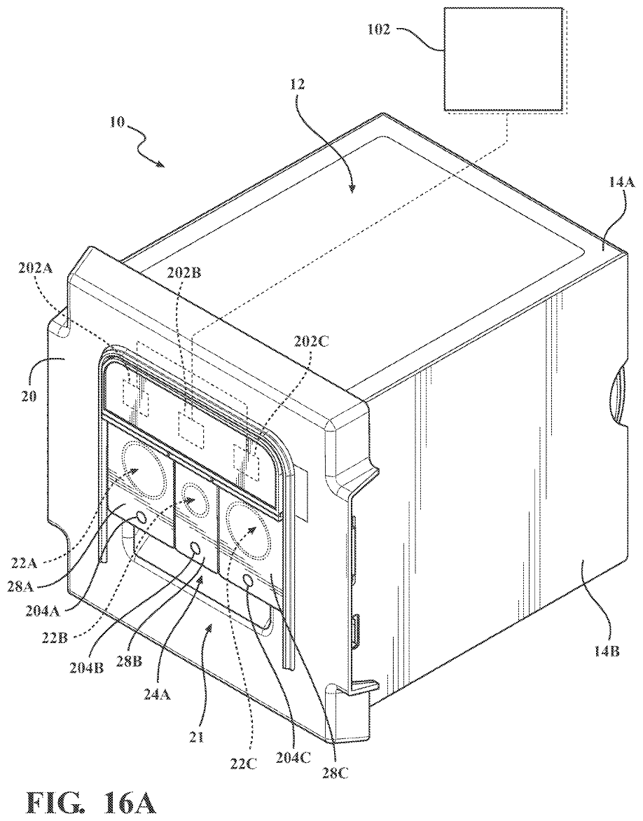

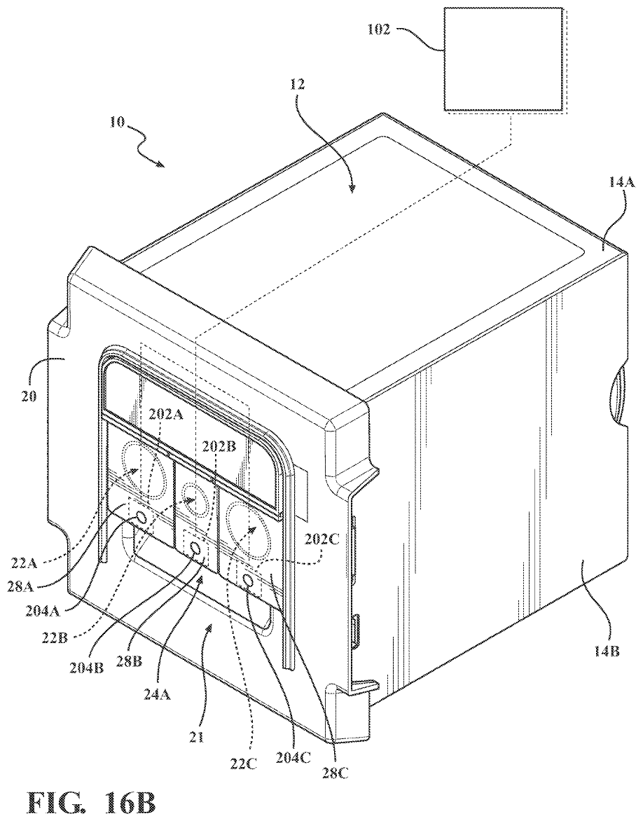

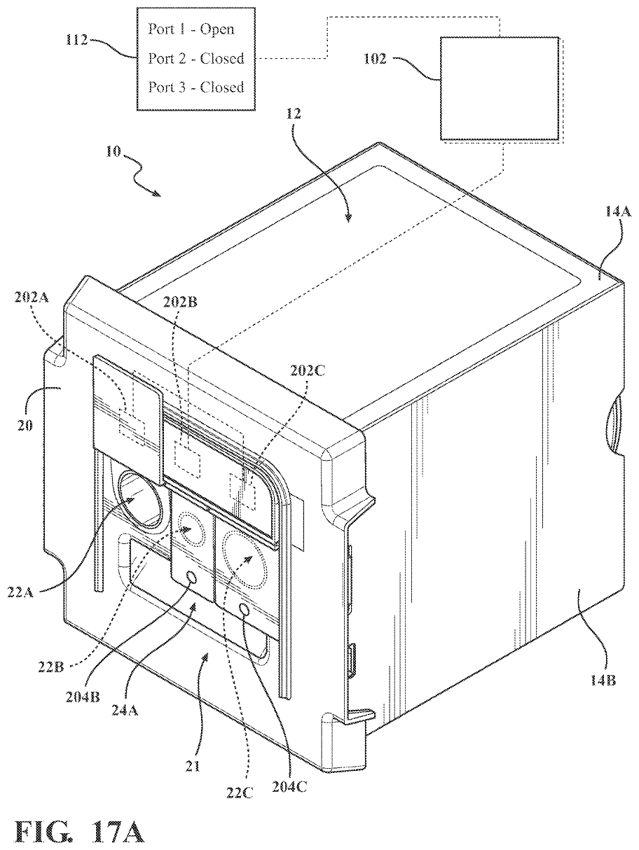

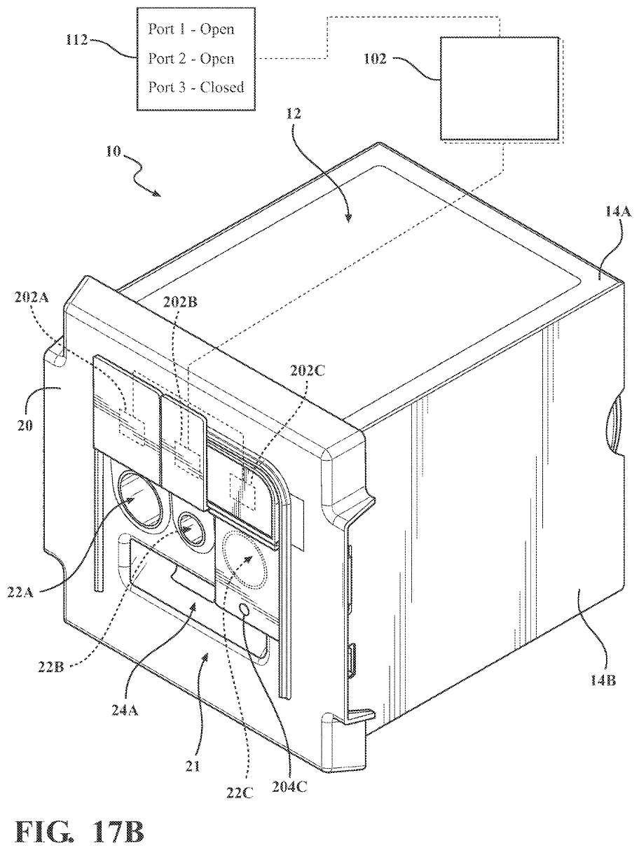

Referring back to FIG. 12, it is further contemplated that the filter assembly 10 of the of the medical waste collection system 100 may optionally comprise a second sensor 202. The second sensor 202 may also be referred to as a flap sensor, connection sensor, port sensor, or the like. The second sensor 202 may be disposed on or within the housing 12 of the filter assembly 10 and configured to identify the position of one or each of the flaps 28A, 28B, 28C for selectively covering the sockets 22A, 22B, 22C of the filter assembly 10. More specifically, the second sensor 202 may be configured to identify when each of the individual flaps 28A, 28B, 28C is in a first position in which the flap 28A, 28B, 28C covers the corresponding socket 22A, 22B, 22C, and when each of the individual flaps 28A, 28B, 28C is in a second position in which the corresponding socket 22A, 22B, 22C is exposed to allow coupling of the smoke evacuation tube with the socket 22A, 22B, 22C. The second sensor 202 may comprise a proximity sensor, hall effect sensor, mechanical switch, optical sensor, or similar sensor configured to identify the presence and/or the absence of an item, such as the flaps 28A, 28B, 28C. For example, the second sensor 202 may comprise a hall effect sensor configured to detect a magnetic field, and each of the flaps 28A, 28B, 28C may comprise an member 204 (also shown in FIGS. 16 to 17B) that is identifiable by the hall effect sensor 202. The member 204 may also be referred to or described as an element, indicia, identifier, tag, component, feature, or the like. The member 204 may comprise and/or be at least partially formed from one of a ferromagnetic material or a magnetic material that it detectable by the hall effect sensor 202. As another example, the member 204 may be formed from a reflective material capable of triggering an optical sensor. In yet another configuration, the member may be formed from a conducive material configured to trigger a capacitive or inductive sensor. This list is not intended to be exhaustive, other combinations of sensors 202 and/or corresponding member 204 capable of identifying the when the flap 28A, 28B, 28C is or is not covering the socket 22A, 22B, 22C are contemplated.

For example, in operation, the second sensor 202, such as a hall effect sensor, may be positioned and/or oriented on or within the housing 12 of the filter assembly 10 such that the hall effect sensor is capable of identifying the position of the flaps 28A, 28B, 28C based on changes in the magnetic field caused by the member of the flaps 28A, 28B, 28C being present or absent. For example, the second sensor 202 may be positioned such that it may detect a first magnetic field when the flap(s) 28A, 28B, 28C is in the first position and covering the corresponding socket 22A, 22B, 22C based on the presence of the member 204 being proximate the second sensor when the flap(s) 28A, 28B, 28C is covering the corresponding socket 22A, 22B, 22C. Alternatively, the second sensor 202 may be positioned such that it may detect a second magnetic field (or an absence of a magnetic field) when the flap(s) 28A, 28B, 28C is in the second position and exposing the corresponding socket 22A, 22B, 22C based on the absence of the member 204 being proximate the second sensor when the flap(s) 28A, 28B, 28C is exposing the corresponding socket 22A, 22B, 22C.

The inverse scenario is also contemplated, wherein the second sensor 202 and/or the member 204 are arranged on or within the housing 12 such the second sensor 202 may detect a second magnetic field (or an absence of a magnetic field) when the flap(s) 28A, 28B, 28C is in the first position and covering the corresponding socket 22A, 22B, 22C based on the absence of the member 204 being proximate the second sensor when the flap(s) 28A, 28B, 28C is covering the corresponding socket 22A, 22B, 22C. in this scenario, the second sensor 202 may then detect a first magnetic field when the flap(s) 28A, 28B, 28C is in the second position and exposing the corresponding socket 22A, 22B, 22C based on the presence of the member 204 being proximate the second sensor when the flap(s) 28A, 28B, 28C is exposing the corresponding socket 22A, 22B, 22C.

The second sensor 202 may further be connected to the controller 102 for controlling one or more operational characteristics of the medical waste collection system 100. The controller 102 of the medical waste collection system 100 may be configured to adjust the settings of the vacuum source 306 (such as a blower assembly) based, at least in part, on the signal received from the second sensor 202.

The medical waste collection system 100 may also comprise a user interface 112. The user interface 112 may comprise a display. The user interface 112 may also include a touch screen, icons, buttons, switches, or a similar user input mechanism for controlling operation of the system 100. The user interface 112 may be connected to the controller 102 and configured for inputting data or instructions to the controller 102 for controlling one or more operational characteristics of the system 100. For example, the user interface 112 may include a button or icon configured to allow the user to input a setting for operation of the vacuum source. The user interface 112 may also include a button or icon for selecting the size, type, and/or number of surgical instruments that are connected to the system 100 via one or more of the sockets 22.

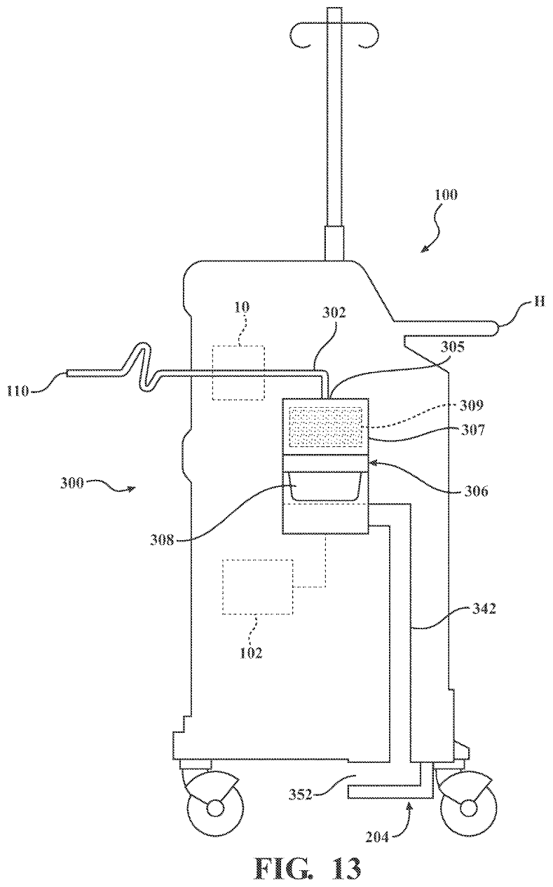

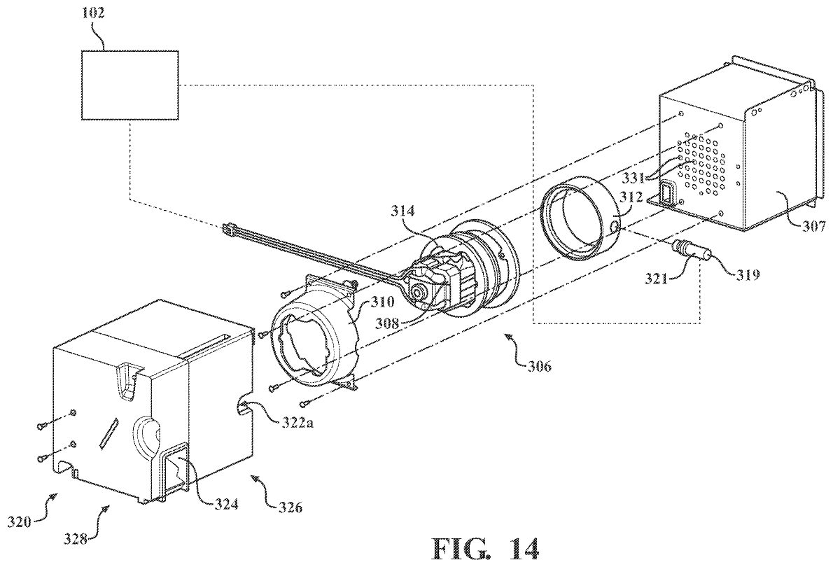

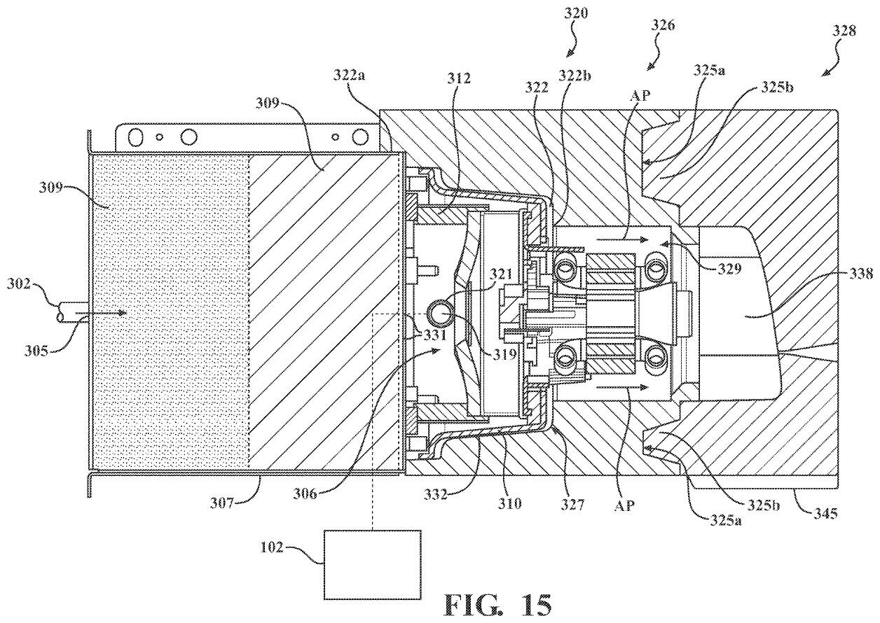

Referring to FIGS. 13 to 15, an example configuration of a vacuum source, a vacuum source 306, of the medical waste collection system 100 is illustrated. The vacuum source 306 may be disposed in a void or compartment 307 defined by the capital component (H). The vacuum source 306 is in fluid communication with the smoke conduit 302 that is connected to the filter assembly 10 (filter assembly shown schematically). The vacuum source 306 draws the fluid into the filter assembly 10 through one or more of the sockets 22A, 22B, 22C when the vacuum source 306 operates. The vacuum source 306 comprises a fan and a blower motor 308 for operating the fan. The vacuum source 306 may comprise a centrifugal fan and the blower motor 308 may be a brush motor. However, those skilled in the art realize alternative embodiments utilizing different implementations of the vacuum source 306.

A sound attenuating enclosure 320, in which the vacuum source 306 is disposed is used to attenuate the noise associated with operation of the vacuum source 306. The sound attenuating enclosure 320 defines an inlet chamber 322 extending between a first enclosure inlet 322a at one end of the sound attenuating enclosure 320 and a second enclosure inlet 322b. The second enclosure inlet 322b receives the filtered air that passes through the exhaust openings 314. The sound attenuating enclosure 320 further defines an enclosure outlet 324 for directing the filtered and warmed air from the vacuum source 306 to the environment.

In addition to the sound attenuating enclosure 320, a muffler 321 may be attached to the centering ring 312 to further attenuate noise associated with the vacuum source 306. During operation of the vacuum source 306, cooling air is drawn through the muffler 321 from inside the waste collection unit 100. More specifically, the muffler 321 defines an inlet 319 for the cooling air to enter the sound attenuating enclosure 320. The inlet 319 may comprise a valve in communication with and/or controlled by the controller 102. The controller 102 may be configured to open and close the valve of the inlet 319 based on the operating conditions of the medical waste collection system 100 to ensure the blower motor is properly cooled. For example, the controller 102 may be configured to open the valve of the inlet 319 when the flap(s) 28A, 28B, 28C are in the first position and covering the corresponding socket 22A, 22B, 22C. When the flap(s) 28A, 28B, 28C are in the first position, air that would otherwise flow from the environment into and through the filter assembly 10 and be used to cool the blower motor 306 is prevented from flowing through the filter assembly 10 and thus is not available to the cool the vacuum source 306. Opening the valve of the inlet 319 provides an alternative pathway for air to enter the sound attenuating enclosure 320 to cool the blowing motor when the flaps 28A, 28B, 28C are in the first position. Inversely, the controller 102 may be configured to close the valve of the inlet 319 when the flap(s) 28A, 28B, 28C are in the second position, exposing the corresponding sockets 22A, 22B, 22C and allowing the flow of air from the environment into and through the filter assembly 10. Air drawn through the sockets 22A, 22B, 22C from the external environment may be used to cool the vacuum source 306. Closing the valve of the inlet 319 when the additional cooling air is not needed via the inlet 319 can provide the advantage of further dampening the sound created by the vacuum source 306 during operation of the system 100. The cooling air provided through the inlet 319 can keep the blower motor 308 cool enough to avoid overheating and/or failure, even if the smoke conduit 302 is occluded and/or all of the flap 28A, 28B, 28C are in the first position and covering the corresponding socket 22A, 22B, 22C preventing the flow of external air into and through the filter assembly 10.

Referring to FIGS. 16A to 17B, a configuration of the filter assembly 10 including the second sensor 202 and member 204 on the flap(s) 28A, 28B, 28C is illustrated. The second sensor and/or member 204 are optional features of the filter assembly 10. It is not a requirement that the filter assembly in one of or either of these features.

As described above, the second sensor 202 may be positioned on or within the filter assembly 10 and configured to identify the position of the flap(s) 28A, 28B, 28C covering the sockets 22A, 22B, 22C defined by the housing 12 and/or the cover 20 of the filter assembly 10. The filter assembly 10 may comprise a single second sensor 202 capable of detecting the position of one or more flap(s) 28A, 28B, 28C. Alternatively, as is illustrated in FIGS. 16A to 17B, the filter assembly may comprise a separate second sensor 202A, 202B, 202C for each of the flap(s) 28A, 28B, 28C of the filter assembly 10.

The member 204 may be any item disposed on and/or coupled to the flap(s) 28A, 28B, 28C that is identifiable and/or detectable by the second sensor 202. For example, as described above, the member 204 may be formed from one of a ferromagnetic material or a magnetic material that it detectable by the hall effect sensor 202. As another example, the member 204 may be formed from a reflective material capable of triggering an optical sensor. In yet another configuration, the member 204 may be formed from a conducive material configured to trigger a capacitive or inductive sensor. The combination of the second sensor 202 and the member 204 may be arranged on the filter assembly 10 such that the presence or absence of the member 204 and detected by the second sensor 202 may be indicative of the position of the flap. For example, as is illustrated in FIG. 16B, the second sensor 202 and the member 204 may be arranged on the filter assembly 10 such that the member 204 is proximate or adjacent the second sensor 202 when the flap(s) 28A, 28B, 28C is in the first position. In this arrangement, the second sensor 202 may be positioned generally below the respective sockets 22A, 22B, 22C such that the detection or identification of the member 204 by the second sensor 202 may indicate the flap(s) 28A, 28B, 28C are covering the respective sockets 22A, 22B, 22C, and the absence of the detection or identification of the member 204 by the second sensor 202 may indicate the flap(s) 28A, 28B, 28C arc in the second position and exposing the respective sockets 22A, 22B, 22C.

Alternatively, as is illustrated in FIG. 16A it is also contemplated that the second sensor 202 and the member 204 may be arranged on the filter assembly 10 such that the member 204 is away from and/or not detectable/identifiable by the second sensor 202 when the flap(s) 28A, 28B, 28C is/are in the first position. In this arrangement, absence of the detection or identification of the member 204 by the second sensor 202 may indicate the flap(s) 28A, 28B, 28C are covering the respective sockets 22A, 22B, 22C, and the detection or identification of the presence of the member 204 by the second sensor 202 may indicate the flap(s) 28A, 28B, 28C are in the second position and exposing the respective sockets 22A, 22B, 22C.

The second sensor 202 may further be configured to produce a signal based on the position of the flap(s) 28A, 28B, 28C. For example, the second sensor 202 may be configured to produce a first signal when the flap(s) 28A, 28B, 28C are determined by the sensor to be in the first position and covering the respective sockets 22A, 22B, 22C. The second sensor 202 may be further be configured to produce a second signal when the flap(s) 28A, 28B, 28C are determined by the sensor to be in the second position and exposing the respective sockets 22A, 22B, 22C. It is also contemplated that the second sensor 202 may be configured to utilize the absence of a signal as an indication of the position of the flap(s) 28A, 28B, 28C. For example, the second sensor 202 may be configured to produce a first signal when the flap(s) 28A, 28B, 28C are in the first position, and produce no signal when the flap(s) 28A, 28B, 28C are in the second position, or vice versa.

The second sensor 202 may be configured to communicate the signal indicative and/or identifying the position of the flap(s) 28A, 28B, 28C to the controller 102. The signal from the second sensor 202 may comprise datum specifically identifying whether the flap(s) 28A, 28B, 28C are in the first position or the second position. For example, the signal from the second sensor 202 may include datum indicating the flap(s) 28A, 28B, 28C is in the first position, and the controller 102 is configured to operate the system 100 based, at least in part, on the flap(s) 28A, 28B, 28C being in the first position. Alternatively, the signal from the second sensor 202 may also be configured to include datum indicating the flap(s) 28A, 28B, 28C is in the second position, and the controller 102 is configured to operate the system 100, based at least in part, on the flap(s) 28A, 28B, 28C being in the second position.

In another configuration of the system 100, the signal from the second sensor 202 may be configured such that the signal may include generic data such as a true/false, yes/no, 1/0, or similar datum based on the flap(s) 28A, 28B, 28C being in the first position or not in the first position, and the controller 102 is further configured to identify the position of the flap based on signal received from the second sensor 202. For example, the second sensor 202 may be configured to send a signal to the controller 102, the single including data indicating “True” when the flap(s) 28A, 28B, 28C are in the first position and the single including data indicating “False” when the flap(s) 28A, 28B, 28C is not in the first position. The controller 102 may then be configured to incorporate the “True” response to indicate the flap(s) 28A, 28B, 28C is in the first position and to operate the system 100 based on the corresponding sockets 22A, 22B, 22C being covered. Alternatively, the controller 102 may be then configured to incorporate a “False” response to indicate the flap(s) 28A, 28B, 28C are not in the first position and to operate the system 100 based on the corresponding sockets 22A, 22B, 22C being exposed.

The controller 102 may further be configured to allow and/or disable operation of the system 100 based on the position of the flap(s) 28A, 28B, 28C. For example, the controller 102 may be configured to disable operation of the vacuum source 306, and by extension the system 100, when all of the flap(s) 28A, 28B, 28C are in the first position and covering the corresponding sockets 22A, 22B, 22C. Referring to FIG. 16, if all flap(s) 28A, 28B, 28C are in the first position and covering the corresponding sockets 22A, 22B, 22C, then there are not exposed sockets 22A, 22B, 22C. The controller 102 can be programmed and/or configured to interpret this to mean that there are no smoke evacuation tubes 110 connected to the system 100, and therefore, operation of the system 100 may be disabled.

Alternatively, when one or more of the flap(s) 28A, 28B, 28C are in the in the second position and exposing the corresponding sockets 22A, 22B, 22C, the controller may be configured to operate the system based on the size of the sockets 22A, 22B, 22C and/or the number of sockets 22A, 22B, 22C that are exposed. For example, the controller 102 may be further configured to control the operation of the system 100 based on the number of sockets 22A, 22B, 22C that are exposed by their corresponding flap(s) 28A, 28B, 28C. In this example, should the second sensor 202A identify the first flap 28A is in the second position exposing the first socket 22A, as seen in FIG. 17A, the controller 102 may be configured to operate the system 100, including the vacuum source 306, at a first volume flow rate. This may be based on the size, shape, etc. of the first socket 22A, which may in turn identify the surgical instrument and/or the size of the smoke evacuation tube 110 that is coupled to the system 100. Alternatively, should the second sensor 202B identify the second flap 28B is in the second position exposing the second socket 22B, the controller 102 may be configured to operate the system 100, including the vacuum source 306, at a second volume flow rate. This again may be based on the size, shape, etc. of the second socket 22B, which may in turn identify the surgical instrument and/or the size of the smoke evacuation tube that is coupled to the system. The system 100 may further be configured such that should the second sensor 202C identify the third flap 28C is in the second position exposing the third socket 22C, the controller 102 may be configured to operate the system 100, including the vacuum source 306, at a third volume flow rate. This again may be based on the size, shape, etc. of the third socket 22B, which may in turn identify the surgical instrument and/or the size of the smoke evacuation tube that is coupled to the system 100. Each of the first, second, and third volume flow rates may be the same or different from one another. Furthermore, the controller 102 may be configured to operate the system 100 a volume rate corresponding to any combination of the first, second and/or third sockets 22A, 22B, 22C being exposed by the corresponding flap(s) 28A, 28B, 28C. For example, the controller 102 may be configured to operate the system 100 at a higher volume flow rate than any of the first, second, or third volume flow rates if multiple sockets 22A, 22B, 22C are exposed by the corresponding flap(s) 28A, 28B, 28C. An example of this is illustrated in FIG. 17B, where a combination of the first and second sockets 22A, 22B are exposed by the corresponding flap(s) 28A, 28B. Multiple sockets 22A, 22B, 22C being exposed by the corresponding flap(s) 28A, 28B, 28C would indicate multiple surgical instruments are coupled to the system 100, and therefore, a high volume flow rate would be required than when only a single instrument is coupled to the system 100.

The user interface 112 may also be configured to identify to the user which of the sockets 22A, 22B, 22C are exposed and/or covered by the corresponding flap(s) 28A, 28B, 28C. For example, as is illustrated in FIG. 17A, the user interface, via the controller 102, may be configured to identify to the user with a visual cue which of the sockets 22A, 22B, 22C are exposed/covered by the corresponding flap(s) 28A, 28B, 28C. This user interface 112 may be configured to identify the exposed/covered sockets 22A, 22B, 22C via text, such as “covered” or “closed” to indicate the flap(s) 28A, 28B, 28C being in the first position and/or “exposed” or “open” to indicate the flap(s) 28A, 28B, 28C being in the second position. Alternatively, the user interface 112 may be configured to identify the position of the flap(s) 28A, 28B, 28C via a color coding system, such as a green light or dot to indicate the flap(s) 28A, 28B, 28C is in the second position and the socket(s) 22A, 22B, 22C is exposed. Alternatively, a red light or dot may be utilized by the user interface 112 to indicate the flap(s) 28A, 28B, 28C is in the first position the socket(s) 22A, 22B, 22C is covered. The specific color for indicating which position the flap is in is immaterial so long as the user understands which color indicates which flap position. It is further contemplated that the user interface 112 may be configured to provide an audible cue identifying which of the sockets 22A, 22B, 22C are exposed/covered by the corresponding flap(s) 28A, 28B, 28C.

A method of operating a filter assembly including a vacuum pump, a flap covering a socket, a sensor, and a controller may comprise the step of receiving, with the controller, a signal from the sensor with the signal indicative that the flap is covering the socket. The method may further comprise the step of preventing, with the controller, operation of the vacuum pump based on the signal. The method may also comprise the step of receiving, with the controller, a second signal from the sensor with the second signal indicative that the flap has been moved away from covering the socket, and enabling, with the controller, operation of the vacuum pump based on the second signal. The method may also comprise the step of displaying, on a user interface, one or more icons for controlling one or more operational characteristics of the vacuum pump based on the second signal being received by the controller.

### Clauses

I. A method of replacing a filter assembly for use with a medical waste collection system including a smoke evacuation tube, the method comprising:
providing the filter assembly, the assembly comprising:
   an housing comprising an opening in the housing, a groove on interior surface of housing, a front cover having an aperture, and a sensor disposed within the housing; and
   a filter cartridge comprising:
      a faceplate having a port to removably couple the smoke evacuation tube to the faceplate;
      an alignment feature coupled to an exterior of the cartridge; and
      a sensor opening in the faceplate;
orienting the filter cartridge so the alignment feature of the filter cartridge may be inserted within the groove on the interior surface of the housing prior to sliding the filter cartridge through the opening;
installing the filter cartridge within the housing by sliding the filter cartridge through the opening so that the alignment feature is disposed within the groove, the sensor is at least partially disposed within the sensor opening of the filter cartridge, and the port of the filter cartridge is aligned with the aperture in the cover; and
installing the filter assembly within the medical waste collection system.

II. The method of clause I, wherein the faceplate further comprises an annular boss encircling the port and extending distally from an outer surface of the faceplate; and
wherein the step of installing the filter cartridge further comprises the step of sliding the filter cartridge through the opening so that the annular boss is at least partially disposed within the aperture in the cover.

III. The method of clause I or II, wherein the step of installing the filter assembly further comprises connecting the sensor to the medical waste collection system.

IV. The method of any one of clauses I to III, further comprising the steps of:
removing the filter assembly from the medical waste collection system;
removing the filter cartridge from the interior of the housing;
orienting a subsequent filter cartridge so that an alignment feature of the subsequent filter cartridge may be inserted within the groove in at least one of the wall members;
installing the subsequent filter cartridge within the housing by sliding the subsequent filter cartridge through the opening so that the alignment feature of the subsequent filter cartridge is disposed within the groove, the sensor is at least partially disposed within an sensor opening of the subsequent filter cartridge, and a port of the subsequent filter cartridge is aligned with the aperture in the cover; and
installing the filter assembly, including the subsequent filter cartridge, within the medical waste collection system.

V. The method of clause IV, further comprising repeating the steps of clause IV following each use of the medical waste collection system to replace the filter cartridge.

VI. The method of clause IV, further comprising the step of counting each occurrence of installing the subsequent filter cartridge within the housing indicative of the number of uses of the filter assembly.

VII. The method of clause IV or VI, further comprising the step of counting each occurrence of installing the filter assembly within the medical waste collection system indicative of the number of uses of the filter assembly; and
indicating when the number of uses of the filter assembly has reached a threshold value to inform the user the filter assembly is due to be replaced.

VIII. The method of clause VI or VII, further comprising the step of providing a visual indicator of the number of occurrences of installing the subsequent filter cartridge within the housing and/or the number of occurrences of installing the filter assembly within the medical waste collection system.

IX. The method of clause I, further comprising the step of counting each occurrence of the step of installing the filter cartridge within the housing by sliding the filter cartridge through the opening indicative of the number of uses of the filter assembly.

X. The method of clause I, further comprising the step of counting each occurrence of the step of installing the filter assembly within the medical waste collection system indicative of the number of uses of the filter assembly; and
indicating when the number of uses of the filter assembly has reached a threshold value to inform the user the filter assembly is due to be replaced.

XI. A filter assembly for use with a medical waste collection system including a smoke evacuation tube coupler, the filter assembly comprising:
an housing defining an interior, the housing comprising a cover;
a sensor coupled to the cover and disposed within the interior of the housing;
a filter cartridge removably disposed within the interior of the housing, the filter cartridge comprising:
   a faceplate;
   a filter portion spaced apart from the faceplate to at least partially define a void space between the faceplate and the filter portion;
   the faceplate comprising a first port in communication with the void space and configured to removably be coupled with the smoke evacuation tube coupler to allow material to pass through the faceplate and enter the void space; and
   the faceplate comprising a sensor opening, the sensor opening configured to receive the sensor when the filter cartridge is disposed within the interior of the housing such that sensor is at least partially disposed within the void space and configured to detect the presence of material entering the void space through the first port.

XII. The filter assembly of clause XI, wherein the filter cartridge further comprises a first alignment feature coupled to a perimeter of the faceplate, the first alignment feature configured to orient the filter cartridge within the interior of the housing.

XIII. The filter assembly of clause XII, wherein the housing comprises a plurality of wall members defining the interior of the housing, and wherein one of the plurality of wall members comprises a groove configured to receive the alignment feature of the filter cartridge when the filter cartridge is disposed within the housing.

XIV. The filter assembly of clause XIII, wherein the filter cartridge further comprising a second alignment feature coupled to the perimeter of the faceplate opposite the alignment feature, wherein a second of the plurality of wall members comprises a second groove configured to receive the second alignment feature of the filter cartridge when the filter cartridge is disposed within the housing, wherein the first alignment feature comprises a first dimension and the second alignment feature comprises a second dimension, and wherein the first dimension is less than the second dimension such that the first dimension and the second dimension are configured to orient the filter cartridge relative to the housing when disposed within the housing.

XV. The filter assembly of clause XI, wherein the faceplate further comprises a second port in communication with the void space and configured to allow material to pass through the faceplate and enter the void space, and wherein the first port comprises a first dimension and the second port comprises a second dimension to allow smoke evacuation tube couplers of different sizes to be coupled to the faceplate.

XVI. The filter assembly of clause XV, wherein each of the first and second ports comprises an annular boss encircling the ports and extending distally from an outer surface of the faceplate, each of the annular bosses configured to create a seal between the smoke evacuation tube coupler and the faceplate to ensure all waste passing through the smoke evacuation tube coupler is collected within the filter cartridge.

XVII. The filter assembly of clause XV, wherein the faceplate comprises a third port, wherein the first, second, and third ports are arranged on the faceplate in a side-by-side linear arrangement, wherein each the first, second and third ports comprise a different dimension configured to receive the smoke evacuation tube couplers of varying sizes, and wherein each of the ports comprises an annular boss encircling the ports and extending distally from an outer surface of the faceplate, the annular boss configured to create a seal between the smoke evacuation tube couplers and the faceplate to ensure all particles passing through the smoke evacuation tube couplers is collected within the filter cartridge.

XVIII. The filter assembly of clause XVII, wherein the cover further comprises a plurality of complementary apertures corresponding to the position of the ports in the faceplate when the filter cartridge is disposed within the housing, and wherein each of the annular bosses encircling the ports is at least partially disposed within one of the plurality of complementary apertures such that a distal end of each of the annular bosses is positioned distally of the cover.

XIX. The filter assembly of clause XVIII, wherein the cover further comprises a flap removably deposed over each of the plurality of complementary apertures of the cover and configured to cover each of the annular bosses that is not coupled to one of the smoke evacuation tube couplers.

XX. The filter assembly of clause XI, further comprising a seal member disposed on the outer surface of the faceplate and encircling the sensor opening, the seal member configured to create a seal between the outer surface of the faceplate and an interior surface of the cover to prevent flow of material from within the void space to the interior of the housing via the sensor opening.

XXI. The filter assembly of clause XI, wherein the filter cartridge comprises a liquid impermeable coating disposed on at least of an interior surface and an exterior surface of the filter cartridge, the liquid impermeable coating configured to prevent liquid from exiting the filter cartridge.

XXII. The filter assembly of clause XI, wherein the first port comprises a cage positioned on an inner surface of the faceplate, the cage extending proximally from the inner surface and at least partially surrounding the first port, and wherein the cage is configured to prevent over insertion of the smoke evacuation tube coupler when coupled to the first port.

XXIII. The filter assembly of clause XI, wherein the filter cartridge further comprises a coupling member disposed on the perimeter of the faceplate, the coupling member configured to removably fasten the filter cartridge within the housing.

XXIV. A filter assembly for use with a medical waste collection system including a smoke evacuation tube coupler, the filter assembly comprising:
an housing defining an interior, the housing comprising a cover;
the cover comprising an aperture defining a pathway from the interior of the housing to an exterior of the housing;
a filter cartridge removably disposed within the interior of the housing, the filter cartridge comprising:
   a faceplate positioned adjacent the cover when the filter cartridge is removably disposed within the interior, the faceplate comprising an inner surface and an opposing outer surface;
   a filter portion spaced apart from faceplate to at least partially define a void space between the inner surface of the faceplate and the filter portion;
   the faceplate comprising a first port in communication with the void space and configured to removably be coupled with the smoke evacuation tube coupler allow material to pass through the faceplate and enter the void space; and
   an annular boss encircling the first port and extending distally from the outer surface of the faceplate, the annular boss configured to create a seal between the smoke evacuation tube coupler and the faceplate;
wherein the annular boss encircling the first port extends through the at aperture in the cover such that a distal end of the annular boss is positioned distally of the cover.

XXV. The filter assembly of clause XXIV, wherein the filter cartridge comprises a liquid impermeable coating disposed on at least of an interior surface and an exterior surface of the filter portion, the liquid impermeable coating configured to prevent liquid from exiting the filter cartridge.

XXVI. The filter assembly of clause XXIV, further comprising a sensor coupled to the cover and disposed within the interior of the housing; and
the faceplate further comprises a sensor opening, the sensor opening configured to receive the sensor when the filter cartridge is disposed within the interior of the housing such that sensor is at least partially disposed within the void space.

XXVII. The filter assembly of clause XXIV, wherein the cover further comprises a flap disposed on an exterior of the cover and configured to cover the annular boss when the smoke evacuation tube coupler is absent.

XXVIII. A filter assembly for use with a medical waste collection system including a smoke evacuation tube coupler, the filter assembly comprising:
an housing defining an interior, the housing comprising a cover including an interior surface and an exterior surface;
a filter cartridge removably disposed within the interior of the housing, the filter cartridge comprising:
   a faceplate comprising an inner surface and an opposing outer surface positioned adjacent the cover when the filter cartridge is removably disposed within the interior, the outer surface comprising a first portion and a second portion;
   a filter portion spaced apart from the faceplate to at least partially define a void space between the inner surface of the faceplate and the filter portion;
wherein the cover comprises a protrusion extending proximally from the interior surface; and
wherein the first portion is positioned distally from the second portion to define a recess in the outer surface of the faceplate to receive the protrusion on the interior surface of the cover when the filter cartridge is removably disposed within the interior of the housing.

XXIX. The filter assembly of clause XXVIII, further comprising:
an aperture in the cover defining a pathway from the interior of the housing to an exterior of the housing;
a first port in the faceplate in communication with the void space and configured to removably be coupled with the smoke evacuation tube coupler to allow material to pass through the faceplate and enter the void space;
an annular boss encircling the first port and extending distally from the outer surface of the faceplate, the annular boss configured to create a seal between the smoke evacuation tube coupler and the faceplate to ensure all particles passing through the smoke evacuation tube coupler are collected within the filter cartridge; and
wherein the annular boss encircling the first port is at least partially disposed within the aperture in the cover such that a distal end of the annular boss is positioned distally of the cover.

XXX. The filter assembly of clause XXVIII, further comprising a sensor coupled to the cover and disposed within the interior of the housing; and
the faceplate further comprises a sensor opening, the sensor opening configured to receive the sensor when the filter cartridge is disposed within the interior of the housing such that sensor is at least partially disposed within the void space.

XXXI. A filter assembly for use with a medical waste collection system including a smoke evacuation tube, the filter assembly comprising:
an housing defining an interior, the housing comprising a cover;
a sensor coupled to the cover and disposed within the interior of the housing;
a filter cartridge removably disposed within the interior of the housing, the filter cartridge comprising:
   a faceplate, a cartridge cover, and a rear plate defining an interior chamber;
   a filter portion disposed within the interior chamber and spaced apart from the faceplate to at least partially define a void space between the faceplate and the filter portion;
   the faceplate comprising a first port in communication with the void space and configured to removably be coupled with the smoke evacuation tube to allow material to pass through the faceplate and enter the void space; and
an absorbent material disposed within at least one of the interior chamber and/or on an interior of the smoke evacuation tube for absorbing liquid substances collected by the smoke evacuation tube to prevent damage internal components of the filter cartridge.

XXXII. A filter cartridge of a filter assembly for use with a medical waste collection system having at least one smoke evacuation tube coupler, the filter assembly including an housing defining an interior and comprising a cover and a sensor coupled to the cover, the filter cartridge comprising:
a faceplate having an inner surface and an opposing outer surface;
a first alignment feature coupled to the faceplate, the first alignment feature configured to orient the filter cartridge within the interior of the housing;
a filter portion having a first end and a second end, the first end spaced apart from the inner surface of the faceplate to at least partially define a void space between the faceplate and the filter portion;
the faceplate comprising a first port and a second port, each configured to receive one of the at least one smoke evacuation tube couplers, each of the ports in communication with the void space and configured to allow smoke to pass through the faceplate and enter the void space, the first port having a first dimension and the second port having a second dimension to allow for removably coupling of different configurations of the at least one smoke evacuation tube coupler to the faceplate; and
the faceplate comprising a sensor opening, the sensor opening configured to receive the sensor when the filter cartridge is disposed within the interior of the housing such that the sensor is at least partially disposed within the void space.

XXXIII. The filter cartridge of clause XXXII, wherein the filter cartridge comprises a liquid impermeable coating disposed on an exterior surface of the filter cartridge, the liquid impermeable coating configured to prevent liquid from entering the filter portion.

XXXIV. The filter cartridge of clause XXXII, wherein the filter cartridge comprises a liquid impermeable coating disposed on an interior surface of the filter cartridge, the liquid impermeable coating configured to prevent liquid from entering the filter portion.

XXXV. The filter cartridge of clause XXXII, wherein the filter cartridge comprises a liquid impermeable coating disposed on an exterior surface and an interior surface of the filter cartridge, the liquid impermeable coating configured to prevent liquid from entering the filter portion.

XXXVI. The filter cartridge of any one of clauses XXXII to XXXV, wherein each of the ports comprises an annular boss that encircles each of the ports and extends distally from the outer surface of the faceplate, the annular boss configured to create a seal between each of the at least one smoke evacuation tube couplers and the faceplate to ensure all particles passing through each of the at least one smoke evacuation tube couplers is collected within the filter cartridge.

XXXVII. The filter cartridge of clause XXXVI, wherein each of the annular bosses extends distally from the outer surface of the faceplate such that a distal end of each of the annular bosses is located distally of the cover when the filter cartridge is disposed within the interior of the housing.

XXXVIII. The filter cartridge of any one of clauses XXXII to XXXVII, wherein each of the ports comprises a cage positioned on to the inner surface of the faceplate, the cage extending proximally from the inner surface and at least partially surrounding each of the ports, and wherein the cage is configured to prevent over insertion of each of the at least one smoke evacuation tube couplers when coupled to one of the ports.

XXXIX. The filter cartridge of any one of clauses XXXII to XXXVIII, wherein the outer surface of the faceplate comprises a first portion and a second portion, and wherein the second portion is positioned proximal of the first portion such that the second portion defines a recess in the faceplate configured to receive a proximally-extending protrusion of the cover of the housing.

XL. The filter cartridge of clause XXXIX, wherein each of the first port, the second port, and the sensor opening are positioned on the first portion of the faceplate such that they are in fluid communication with a portion of the void space where the distance between the filter portion and the inner surface of the faceplate is greatest.

XLI. The filter cartridge of any one of clauses XXXII to XL, wherein the filter cartridge further comprises a coupling member disposed on a perimeter of the faceplate, the coupling member configured to removably fasten the filter cartridge within the housing.

XLIII. The filter cartridge of any one of clauses XXXII to XLI, wherein the faceplate comprises a third port, wherein the first, second, and third ports are arranged on the faceplate in a side-by-side linear arrangement, wherein each the first, second and third ports comprise a different dimension configured to receive one of the at least one smoke evacuation tube couplers wherein each of the at least one smoke evacuation tube couplers vary in size, and wherein each of the ports comprises an annular boss encircling the ports and extending distally from the outer surface of the faceplate, the annular boss configured to create a seal between each of the at least one smoke evacuation tube couplers and the faceplate.

XLIV. The filter cartridge of any one of clauses XXXII to XLIII, wherein the faceplate further comprises a valve at least partially disposed within each of the ports configured to prevent backflow of particles entering the void space through the ports.

XLV. The filter cartridge of any one of clauses XXXII to XLIV, further comprising a second alignment feature coupled to the faceplate,
wherein the first alignment feature comprises a first dimension and the second alignment feature comprises a second dimension,
wherein the first dimension is less than the second dimension such that the first dimension and the second dimension are configured to orient the filter cartridge relative to the housing when disposed within the housing.

XLVI. The filter cartridge of clause XLV, wherein the second alignment feature is diametrically opposed to the first alignment feature.

XLVII. A filter cartridge for a filter assembly for filter smoke, the filter assembly including a filter assembly housing including a front cover at least partially defining an interior, and a sensor assembly including a sensor housing and a sensor, the filter cartridge comprising:
a faceplate configured to be positioned within the interior of the filter assembly housing near the front cover with the faceplate comprising an outer surface opposite an inner surface and defining a port configured to be removably coupled with a smoke evacuation tube, and a sensor opening separate from the port with the sensor opening sized to receive at least a portion of the sensor housing of the sensor assembly;
a filter portion comprising a front, a rear opposite the front, and a side extending between the front and the rear with the front directed towards the inner surface of the faceplate;
the faceplate and the filter portion are spaced apart from one another to at least partially define a void space between the inner surface of the faceplate and the front of the filter portion,
wherein the sensor opening and the port are complementarily arranged and in communication with the void space such that, when the filter cartridge is disposed within the interior of the filter assembly housing and the smoke evacuation tube is removably coupled with the port, the sensor is positioned within the void space to detect smoke within the void space received from the smoke evacuation tube through the port before encountering the filter portion.

XLVIII. The filter cartridge of clause XLVII, wherein the port comprises an annular boss that encircles the port and extends distally from the outer surface of the faceplate, the annular boss configured to create a seal between the smoke evacuation tube and the faceplate to ensure all particles passing through the smoke evacuation tube is collected within the filter cartridge.

XLIX. The filter cartridge of clause XLVIII, wherein the annular boss extends distally from the outer surface of the faceplate such that a distal end of the annular boss is located distally of the cover when the filter cartridge is disposed within the housing.

L. The filter cartridge of any one of clauses XLVII to XLIX, wherein the port comprises a cage positioned on to the inner surface of the faceplate, the cage extending proximally from the inner surface and at least partially surrounding the port, and wherein the cage is configured to prevent over insertion of the smoke evacuation tube when coupled to the port.

LI. The filter cartridge of any one of clauses XLVII to L, wherein the outer surface of the faceplate comprises a first portion and a second portion, and wherein the second portion is positioned proximal of the first portion such that the second portion defines a recess in the faceplate configured to receive a protrusion of the cover of the housing.

LII. A method of replacing a filter assembly for use with a medical waste collection system including a smoke evacuation tube, the method comprising:
providing the filter assembly, the assembly comprising:
   an housing comprising an opening in the housing, a groove on interior surface of housing, a front cover having an aperture, and a sensor disposed within the housing; and
   a filter cartridge comprising:
      a faceplate having a port to removably couple the smoke evacuation tube to the faceplate;
      an alignment feature coupled to an exterior of the cartridge; and
      a sensor opening in the faceplate;
orienting the filter cartridge so the alignment feature of the filter cartridge may be inserted within the groove on the interior surface of the housing prior to sliding the filter cartridge through the opening;
installing the filter cartridge within the housing by sliding the filter cartridge through the opening so that the alignment feature is disposed within the groove, the sensor is at least partially disposed within the sensor opening of the filter cartridge, and the port of the filter cartridge is aligned with the aperture in the cover; and
installing the filter assembly within the medical waste collection system.

LIII. A method of operating a filter assembly including a vacuum pump, a flap covering a socket, a sensor, and a controller, the method comprising:
receiving, with the controller, a signal from the sensor that is indicative that the flap has been moved and the socket is exposed; and
enabling, with the controller, operation of the vacuum pump based on the signal.

LIV. The method of clause LIII, further comprising the step of operating the vacuum pump based on the signal from the sensor, the signal further indicative of a size of the socket that has been exposed by moving the flap to uncover the socket.

LV. The method of clause LIII, wherein the filter assembly comprises a plurality of sockets with a flap covering each of the plurality of sockets; and
further comprising the step of operating the vacuum pump based on the signal from the sensor, the signal further indicative of a quantity of the plurality of sockets that have been exposed by moving the flap to uncover the corresponding socket.

LVI. The method of clause LIV, wherein the filter assembly further comprises a user interface and the method further comprises the step of enabling or disabling one or more features of the user interface based, at least in part, on the size of the socket that is exposed by moving the flap to uncover the socket.

LVII. The method of any one of clauses LIII to LVI, further comprising the step of:
receiving, with the controller, a second signal from the sensor with the second signal indicative that the flap is covering the socket; and
disabling, with the controller, operation of the vacuum pump based on the second signal.

Several embodiments have been discussed in the foregoing description. However, the embodiments discussed herein are not intended to be exhaustive or limit the filter assembly 10, housing 12, and/or the filter cartridge 38 to any particular form. The terminology which has been used is intended to be in the nature of words of description rather than of limitation. Many modifications and variations are possible in light of the above teachings and the system may be practiced otherwise than as specifically described.

1. A system for evacuating surgical smoke, the system comprising:
a capital component defining a receiver and comprising a vacuum source, and a controller in electronic communication with the vacuum source;
a filter assembly comprising:
a housing configured to be removably disposed within the receiver of the capital component, the housing defining a first socket for removably receiving a smoke evacuation tube;
a filter disposed within the housing;
a first flap coupled to the housing and configured to be moved from a first position in which the first flap covers the first socket to a second position in which the first socket is exposed to allow coupling of the smoke evacuation tube with the first socket; and
a sensor configured to be arranged in electronic communication with the controller with the filter assembly coupled to the capital component, wherein the sensor is coupled to the housing or the first flap and configured to detect the first flap in the first position,
wherein the controller is configured to disable operation of the vacuum source based on the sensor detecting the first flap in the first position.

2. The system of claim 1, further comprising a valve associated with the vacuum source, the valve moveable between a closed position in which the void is sealed from an external environment and an open position in which the void is exposed from an external environment, wherein the controller is further configured to move the valve from the closed position to the open position based on the sensor detecting the first flap in the first position.

3. The system of claim 1, further comprising an member disposed on the first flap, the member is at least partially formed from one of a magnetic material or a ferromagnetic material, wherein the sensor comprises a hall-effect sensor configured to detect changes in a magnetic field based on presence or absence of the member to determine the position of the first flap.

4. A system for evacuating surgical smoke comprising:
a housing defining a first socket for removably receiving a smoke evacuation tube;
a filter disposed within an interior of the housing;
a vacuum source configured to draw suction on the first socket to draw fluid through the filter;
a first flap coupled to the housing and configured to be moved from a first position in which the first flap covers the first socket to a second position in which the first socket is exposed to allow coupling of the smoke evacuation tube with the first socket;
a sensor coupled to the housing and configured to detect the first flap in the first position;
a user interface; and
a controller in electronic communication with the sensor and the user interface, the controller configured to determine the first flap is in a second position based on an absence of a signal being received from the sensor and to cause the user interface to display information based on the absence of the signal.

5. The system of claim 1, further comprising a user interface; and
wherein the controller is further configured to cause the user interface to display information including one or more icons for controlling one or more operational characteristics of the vacuum source on the user interface based on the presence or absence of a signal from the sensor indicative that the first flap is in the second position.

6. The system of claim 1, wherein the controller is further configured to disable operation of the vacuum source based on the sensor detecting the first flap in the first position, and present an alert for the user to insert or reinsert the smoke evacuation tube into the first socket.

7. The system of claim 1, further comprising:
a user interface including a display;
a second socket defined by the housing; and
a second flap coupled to the housing and configured to be moved from a first position in which the second flap covers the second socket to a second position in which the second socket is exposed to allow coupling of the smoke evacuation tube with the second socket,
wherein the controller is further configured to:
cause the display, on the user interface, of a first configuration of icons with the first flap is in the second position and the second flap is in the first position; and
cause the display, on the user interface, of a second configuration of icons with the second flap is in the second position and the first flap is in the first position.

8. The system of claim 1, further comprising:
a second socket defined by the housing; and
a second flap coupled to the housing and configured to be moved from a first position in which the second flap covers the second socket to a second position in which the second socket is exposed to allow coupling of the smoke evacuation tube with the second socket,
wherein the controller is further configured to operate the vacuum source:
at a first volume flow rate when the first flap is in the second position and the second flap is in the first position, indicative of the first socket having a first socket dimension being exposed;
at a second volume flow rate when the first flap is in the first position and the second flap is in the second position, indicative of the second socket having a second socket dimension being exposed; and
at a third volume flow rate when the first flap is in the second position and the second flap is in the second position, indicative of the first socket having the first socket dimension being exposed and the second socket having the second socket dimension being exposed.

9. The system of claim 1, further comprising a user interface in electronic communication with the controller; and
the controller further configured to:
display, on a user interface, a first configuration of buttons for manual manipulation by a user for controlling one or more operational characteristics of the vacuum source when the first flap is in the second position and the second flap is in the first position; and
display, on the user interface, a second configuration of buttons for manual manipulation by a user for controlling one or more operational characteristics of the vacuum source when the second flap is in the second position and the first flap is in the first position.

10. The system of claim 8, wherein the controller is further configured to disable operation of the vacuum source based on the sensor detecting both the first flap and the second flap being in the first position.

11.-16. (canceled)

17. A filter assembly for use with capital equipment including a vacuum source, the filter assembly comprising:
a housing defining a first socket for removably receiving a smoke evacuation tube;
a filter disposed within an interior of the housing;
a first flap coupled to the housing and configured to be moved from a first position in which the first flap covers the first socket to a second position in which the first socket is exposed to allow coupling of the smoke evacuation tube with the first socket; and
a sensor coupled to the housing or the first flap and configured to identify the position of the first flap relative to the first socket.

18. The filter assembly of claim 17, further comprising:
a member disposed on the first flap, the member at least partially formed from one of a magnetic material or a ferromagnetic material and configured to be detectable by the sensor to determine the position of the first flap;
a fastener disposed on the housing proximate the first socket, the fastener at least partially formed from the other of a ferromagnetic material or a magnetic material, the fastener magnetically attracted to the member disposed on the first flap; and
wherein the sensor comprises a hall-effect sensor configured to detect changes in a magnetic field proximate the fastener based on the position of the first flap and generate a signal indicative of the first flap being in either the first position or the second position based on the magnetic field.

19.-21. (canceled)

22. The filter assembly of claim 17, the filter assembly further comprising:
a second socket having an inner diameter different than the first socket;
a second flap configured to be moved from a first position in which the first flap covers the second socket to a second position in which the second socket is exposed; and
a second sensor positioned on one of the housing or the second flap.

23. The filter assembly of claim 17, wherein the housing further comprises a cover removably coupled to the housing, the cover configured to provide access to the interior of the housing for removal and insertion of the filter.

24.-26. (canceled)

27. The filter assembly of claim 17, wherein the sensor is positioned above the first socket define by the housing and the sensor is configured to detect when the first flap is in the second position; and
wherein the sensor is configured to identify the first flap is in the first position based on the absence of the sensor detecting the first flap is in the second position.

28. (canceled)

29. The filter assembly of claim 17, wherein the sensor is positioned below the first socket define by the housing and the sensor is configured to detect when the first flap is in the first position; and
wherein the sensor is configured to identify the first flap is in the second position based on the absence of the sensor detecting the first flap is in the first position.

30.-40. (canceled)

41. The system of claim 4, further comprising:
a second socket defined by the housing; and
a second flap coupled to the housing and configured to be moved from a first position in which the second flap covers the second socket to a second position in which the second socket is exposed to allow coupling of the smoke evacuation tube with the second socket,
wherein the controller is further configured to operate the vacuum source:
at a first volume flow rate when the first flap is in the second position and the second flap is in the first position, indicative of the first socket having a first socket dimension being exposed;
at a second volume flow rate when the first flap is in the first position and the second flap is in the second position, indicative of the second socket having a second socket dimension being exposed; and
at a third volume flow rate when the first flap is in the second position and the second flap is in the second position, indicative of the first socket having the first socket dimension being exposed and the second socket having the second socket dimension being exposed.

42. The system of claim 41, wherein the controller is further configured to disable operation of the vacuum source based on the sensor detecting both the first flap and the second flap being in the first position.

43. The system of claim 41, further comprising:
a capital component defining a void, the vacuum source at least partially disposed within the void;
a valve disposed on the capital component, the valve moveable between a closed position in which the void is sealed from an external environment and an open position in which the void is exposed from an external environment; and
wherein the controller is further configured to move the valve from the closed position to the open position based on the sensor detecting both the first flap and the second flap being in the first position.

44. The system of claim 4, further comprising:
a second socket defined by the housing; and
a second flap coupled to the housing and configured to be moved from a first position in which the second flap covers the second socket to a second position in which the second socket is exposed to allow coupling of the smoke evacuation tube with the second socket,
wherein the controller is further configured to:
cause the display, on the user interface, of a first configuration of icons with the first flap is in the second position and the second flap is in the first position; and
cause the display, on the user interface, of a second configuration of icons with the second flap is in the second position and the first flap is in the first position.
